(19) United States
(12) Patent Application Publication
(10) Pub. No.: US 2023/0340624 A1
(43) Pub. Date: Oct. 26, 2023

(54) DETECTION REAGENTS FOR SEVERE ACUTE RESPIRATORY SYNDROME CORONAVIRUS 2 AND DETECTION METHODS

(71) Applicant: Zhejiang Orient Gene Biotech Co.,Ltd, Anji Huzhou, Zhejiang (CN)

(72) Inventors: Yeqi TAO, Anji Huzhou, Zhejiang (CN); Xingzhong GUO, Anji Huzhou, Zhejiang (CN); Wen CHEN, Anji Huzhou, Zhejiang (CN); Jianqiu FANG, Anji Huzhou, Zhejiang (CN)

(21) Appl. No.: 17/909,217

(22) PCT Filed: Mar. 4, 2021
(86) PCT No.: PCT/CN2021/079164

§ 371 Date: Sep. 2, 2022

(30) Foreign Application Priority Data
Mar. 5, 2020 (CN) 202010147912.7
Aug. 13, 2020 (CN) 202010814021.2

(51) Int. Cl. C12Q 1/70

(52) U.S. Cl. CPC C12Q 1/701

(57) ABSTRACT

The present invention provides a kit for quantitatively detecting a copy number of Severe acute respiratory syndrome coronavirus 2 (SARS-CoV-2). The kit includes a nucleic acid extraction kit and a nucleic acid amplification kit. The nucleic acid extraction kit adopts a magnetic bead method to perform rapid nucleic acid lysis and adsorption on a sample. The nucleic acid amplification kit adopts a fluorescence quantitative RT-PCR technology for quantitative analysis. Meanwhile, optimal primer and probe sequences are designed, and a pseudotype virus containing SARS-CoV-2 and HCV gene fragments is used as a standard. A SARS-CoV-2 content of a pseudotype virus stock solution is subjected to calibrated detection by HCV with an international standard, so that a copy number of the standard is characterized very accurately, which overcomes inaccuracy of relative judgment standards of a CT value by fluorescent PCR analysis, further improves an effect of nucleic acid detection of the SARS-CoV-2, and improves detection sensitivity. The kit for quantitatively detecting the copy number of the SARS-CoV-2 provided by the present invention has relatively high sensitivity and specificity, can accurately quantify the copy number of the SARS-CoV-2, is easy and convenient to operate, and can provide accurate and reliable results.

### REFERENCE TO AN ELECTRONIC SEQUENCE LISTING

The content of the electronic sequence listing (2023-07-06-Sequence-Lising.txt; Size: 14,612 bytes; and Date of Creation: Jul. 6, 2023) is herein incorporated by reference in its entirety.

### CROSS REFERENCE TO RELATED APPLICATIONS

This application claims the priority of the prior Chinese application with the application number of 2020101479127 applied on Mar. 5, 2020; and the prior Chinese application with the application number of 2020108140212 applied on Aug. 13, 2020, and all the contents of this application are regarded as a part of the present invention.

### TECHNICAL FIELD

The present invention relates to the fields of biotechnology and medicine, in particular to a method and a kit for detecting Severe acute respiratory syndrome coronavirus, which are suitable for detection and clinical auxiliary diagnosis of Severe acute respiratory syndrome coronavirus infection.

### BACKGROUND

Coronavirus disease 2019 (COVID-19) is a disease caused by Severe acute respiratory syndrome coronavirus 2 (SARS-CoV-2). In December 2019, patients with pneumonia of unknown cause appeared in succession in some medical institutions in Wuhan. Wuhan continued to carry out monitoring of influenza and related diseases. On Jan. 7, 2020, a laboratory detected a novel coronavirus, and obtained a whole genome sequence of the virus. At present, a source of infection is mainly patients infected with the SARS-CoV-2, and asymptomatic patients may also become the source of infection. Modes of transmission are mainly respiratory droplet transmission and contact transmission, and the population is generally susceptible. The SARS-CoV-2 belongs to the genus β coronavirus. Evolutionary analysis shows that the SARS-CoV-2 is most similar to bat severe acute respiratory syndrome-related coronaviruses from Rhinolophus sinicus (a species of Chinese horseshoe bat), a nucleotide homology thereof reaches 84%, a nucleotide homology with human SARS viruses reaches 78%, and a homology with MERS viruses reaches about 50%. At present, there are no clear and effective drugs and vaccines against the SARS-CoV-2, so control over the further development of epidemic mainly depends on timely detection and isolation.

With the rapid development of molecular biology diagnosis technology in recent years, a fluorescence quantitative PCR technology has become an important means of increasing attention in genetic diagnosis of infectious disease pathogens. The fluorescence quantitative PCR (FQ-PCR) technology is a quantitative PCR technology that is widely used at home and abroad currently. It is realized by real-time monitoring of cumulative fluorescence intensity through the initial point quantification and fluorescence detection systems. The FQ-PCR technology labeled with a TaqMan probe method is most widely used. This technology overcomes the detection difficulty of traditional detection technology due to the relatively low content of pathogens in samples, and can determine the type of infectious disease pathogens within a few hours. It has high sensitivity, strong specificity, good linear relationship, simple operation and high degree of automation, pollution resistance, high throughput, rapidness and other characteristics, and has been widely used in qualitative and quantitative detection of human and animal infectious diseases. In RT-PCR detection of RNA viruses, there are many factors that affect a test process, such as operation errors, equipment errors, reagent differences, and differences in processing of samples themselves, which may cause different test results, and may cause false negatives and positives. Therefore, RT-PCR determination must use quality control products for strict quality control, so that reliability of results can be ensured. In addition, for quantitative determination, there must be a quantitative standard or internal standard. Due to the potential infectious risk of natural viruses and the instability of RNA molecules thereof, currently used quality control products such as inactivated virus particles, cDNA, plasmid DNA, naked RNA and in vitro transcribed RNA are difficult to meet test requirements. Therefore, the development of stable, reliable and non-biologically infectious quality control materials and standards is of great significance not only for the RT-PCR detection of the RNA viruses, but also for the evaluation of commercial viral RT-PCR kits.

In this context, preparation of standards by a pseudotype virus packaging technology will be a very safe and reliable research means. A pseudotype virus refers to that a virus has its own genetic material, but an envelope thereof is coated with a glycoprotein of another virus, so it has infection characteristics of another virus. The pseudotype virus is equally invasive to host cells as an euvirus that provide envelope proteins. This phenomenon of inconsistent genotype and phenotype is called pseudotype, and the virus with this characteristic is called a pseudotype virus. This virus can only carry out “one cell cycle” infection, and biological safety thereof is relatively strong. Based on a retroviral vector, a foreign gene to be packaged is combined with a reporter gene to be transfected, so as to integrate the foreign gene into a host cell genome to achieve the purpose of stable expression (establishing a cell line) or transient expression. Through the replication of a retrovirus and a packaging signal provided by the vector, a replication-deficient pseudotype virus particle in which a foreign gene is encapsulated by a retrovirus capsid can be obtained. The use of the pseudotype virus particle containing a target RNA as a quality control product and standard for RNA virus fluorescence quantitative RT-PCR detection not only has no risk of biological infection, but also has a good simulation effect on RNA in pathogens in nucleic acid extraction. Meanwhile, a pseudotype virus RNA is not prone to cause cross-contamination of experimental instruments and the environment and result in false positive results, thereby avoiding the problem that commonly used plasmid standards in commercial kits previously developed cannot process samples well and cannot effectively control a reverse transcription process. Therefore, as a positive reference for a kit, it is a very good solution to construct a positive reference that can simulate the whole process from RNA to nucleic acid amplification of a virus.

CN111378785A discloses that a pseudotype virus constructed by using RdRp, Gene E and Gene N genes of the SARS-CoV-2 as a standard, and q-PCR is used for quantitatively detecting the SARS-CoV-2. However, because there is no international standard for the SARS-CoV-2 at present, it is still difficult to accurately quantify a copy number of the SARS-CoV-2 by using a pseudotype virus containing an SARS-CoV-2 gene; because the RNA of the pseudotype virus needs to be extracted to be reverse-transcribed into DNA, PCR reaction is further conducted; in addition, it is necessary to use detection systems of primers and probes of the CDC and WHO to carry out absolute quantification of the copy number of a target gene of the pseudotype virus by ddPCR, which is then used in performance evaluation of kits for nucleic acid diagnosis of 2019-nCoV, resulting in a lot of inconvenience, and there is still a risk of imprecise quantification of the copy number of the SARS-CoV-2.

The nucleic acid extraction process of current COVID-19 nucleic acid detection kits on the market is complex, long time-consuming, and has low sensitivity, making it difficult to accurately quantify the copy number of the SARS-CoV-2. Moreover, at present, there is no relevant report on magnetic bead nucleic acid extraction and RT-PCR quantitative detection of the SARS-CoV-2, and kits for accurately quantifying the copy number of the SARS-CoV-2.

On the other hand, at present, there are thousands of fluorescent PCR detection reagents and methods that have been used clinically; a conventional method for detecting pathogen nucleic acids by fluorescent PCR generally requires two independent processes, one of the two independent processes is preparation of a pathogen DNA or RNA nucleic acid template, and the other is to add the nucleic acid template to a reaction system for PCR amplification detection. Due to different structures of various pathogens, nucleic acid processing reagents and methods used and established in various laboratories are not completely consistent. However, treatment processes are basically the same, and all need multiple steps such as digestion and cracking, adsorption purification, washing and collection. The processing of a single sample usually takes tens of minutes, and when a large number of samples need to be processed, it takes even longer time than PCR detection reaction. Due to numerous operation steps, mutual contamination between samples is unavoidable during processing of large samples; due to improper protection, it is more common for humans to be infected with pathogens during processing.

Therefore, it is hoped to provide a reagent or a detection method with higher detection sensitivity and a simpler processing process for sample processing, and at the same time to achieve comprehensive detection of mutated viruses without leaked detection.

### SUMMARY

Generally, an extraction-free fluorescent PCR technology adopts an optimized PCR buffer system and a special DNA polymerase to reduce the interference of various inhibitors in a sample on PCR reaction, and has the characteristics of strong activity, high stability, strong tolerance, etc. The technology of the present invention does not need to perform an expensive and time-consuming nucleic acid extraction process, and can be directly used for fluorescent PCR amplification identification by using animal specimens or pathogen preservation solutions, which greatly simplifies operation steps and shortens detection time.

In a first aspect, the present invention aims to provide a method and a kit for extraction-free nucleic acid detection of Severe acute respiratory syndrome coronavirus 2 (SARS-CoV-2), which simplifies operation steps and shortens PCR detection time.

In order to achieve the above purpose, the technical solution of the present invention is to design two pairs of specific primers and fluorescent probes for specific conserved regions of two different gene coding regions of the SARS-CoV-2 as target regions, and meanwhile set up an internal standard quality control system for quality control of an entire reaction system.

Therefore, in some implementations, a kit for extraction-free nucleic acid detection of Severe acute respiratory syndrome coronavirus 2 (SARS-CoV-2) includes an nCoV reaction solution, a PCR enhancer, an nCoV positive quality control product and an nCoV negative quality control product.

The nCoV reaction solution in the kit for detection of the SARS-CoV-2 includes a primer probe, a DNA polymerase, a reverse transcriptase, a UNG enzyme, a nucleoside triphosphate, magnesium ions, Tris-HCl, KCl, MgCl₂ and TritonX-100.

Specific primers and probes in the nCoV reaction solution are primers and probes designed for conserved sequences of an ORF1ab gene and an N gene of the SARS-CoV-2, as well as primers and probes for a β-Globin gene as an internal standard.

In some implementations, sequences of ORF1ab gene-specific forward and reverse primers are respectively 5′-GGCTTCACATATGTATTGTTC-3′ (SEQ IN NO: 34) and 5′-GCTCAAACTCTTCTTCTTCAC-3′ (SEQ IN NO: 35); a sequence of an ORF1ab gene-specific probe is 5′-TCACCTTCTTCTTCATCCTCATCTGG-3′ (SEQ IN NO: 36), and two ends of the probe are respectively labeled with a fluorescence generating group FAM and a fluorescence quenching group BHQ1.

In some implementations, sequences of N gene-specific forward (upstream) and reverse (downstream) primers are respectively 5′-AAGGCTTCTACGCAGAAG-3′ (SEQ IN NO: 37) and 5′-GCTGCCTGGAGTTGAATTTC-3′ (SEQ IN NO: 38); a sequence of an N gene-specific probe is 5′-AGCCTCTTCTCGTTCCTCATCAC-3′ (SEQ IN NO: 39), and two ends of the probe are respectively labeled with a fluorescence generating group ROX and a fluorescence quenching group BHQ2.

In some implementations, sequences of β-Globin gene-specific forward and reverse primers are respectively 5′-CTGAGGGTTTGAAGTCCA-3′ (SEQ IN NO: 40) and 5′-TCTGCCCTGACTTTTATG-3′ (SEQ IN NO: 41); a sequence of a β-Globin gene-specific probe is 5′-CTCCTAAGCCAGTGCCAGAAGA-3′ (SEQ IN NO: 42), and two ends of the probe are respectively labeled with a fluorescence generating group VIC and a fluorescence quenching group BHQ1.

An optimal concentration of the DNA polymerase in the nCoV reaction solution in the kit for detection of the SARS-CoV-2 is 2 U/reaction, a usage amount of the reverse transcriptase is 1.5 U/reaction, and a usage amount of the UNG enzyme is 0.2 U/reaction; an optimal concentration of dNTPs is 2 mmol/L, and 5 mmol/L Tris-HCl, 10 mmol/L MgCl₂ and 30 mmol/L KCl are further included.

The PCR enhancer in the kit for detection of the SARS-CoV-2 includes 1% to 6% formamide, 0.01% to 0.2% glycerol, and 1 to 5 mg/ml BSA, functioning to improve activity of the DNA polymerase and increase heat resistance of the reverse transcriptase.

In some implementations, the nCoV reaction solution also includes a PCR enhancer, so that all reaction solutions are stored in one tube of reagent. In addition, an nCoV positive quality control product and an nCoV negative quality control product are included.

In some implementations, the positive quality control product is a pUC57 vector plasmid containing an inserted SARS-CoV-2-specific conserved sequence. The negative quality control product is a TE buffer containing a human house-keeping gene β-Globin gene sequence.

In a second aspect, in some implementations, the present invention provides a method for detecting Severe acute respiratory syndrome coronavirus 2 (SARS-CoV-2), including as follows.

### 1. Preparation of Reaction System

A PCR enhancer is added according to the number of reactions, with 4 μL for each reaction, and an nCoV reaction solution is 35 μL. According to 39 μL/reaction, a PCR reaction solution is subpackaged into PCR tubes added with samples, lids are closed, and the PCR tubes are transferred to a PCR amplification area. The nCoV reaction solution includes a primer probe, a DNA polymerase, a reverse transcriptase, a UNG enzyme, a nucleoside triphosphate, magnesium ions, Tris-HCl, KCl, MgCl₂ and TritonX-100. The enhancer is prepared from a mixed liquid of 1% to 6% formamide, 0.01% to 0.2% glycerol, and 1 to 5 mg/ml BSA. Of course, it can be understood that before reaction is needed, the nCoV reaction solution is mixed with an enhancer solution to form a final reaction liquid.

### 2. Sample Processing

(1) a throat swab or a nasal swab (a cotton swab or a flocked swab) containing a patient specimen is washed in 200 to 300 μL of normal saline (or a virus preservation solution); and
(2) 11 μL of a swab washing solution (if it is a sputum sample, vortex oscillation is need for more than 20 seconds, and 11 μL thereof is sucked by avoiding a viscous liquid) is sucked, or 11 μL of the virus preservation solution is added into a PCR reaction tube; meanwhile, positive quality control and negative quality control should be performed in parallel.

### 3. Fluorescent PCR Amplification and Detection

Reaction conditions:

| | Number of | Temperature | Reaction time |
|---|---|---|---|
| Steps | cycles | (° C.) | (min:sec) |
| 1 | 1 | 50 | 15:00 |
| 2 | 1 | 96 | 01:00 |
| | | 96 | 00:03 |
| 3 | 45 | 58 | 00:12 |

### 4. Result Interpretation

After reaction is over, an instrument automatically saves results. After images are analyzed, a Start value, an End value and a Threshold value of Baseline are adjusted (they can be self-adjusted, the Start value can be between 3 and 15, the End value can be between 5 and 20, an amplification curve of a negative quality control product is adjusted to be flat or below a threshold line), a Ct value displayed by software is read, and the following analysis is done.

Various fluorescence channels are selected respectively to read Ct values. Judgment is conducted by referring to Table 1 below.

| | FAM fluorescence | |
|---|---|---|
| | | Ct > 38.5 or |
| Ct value | Ct ≤ 38.5 | no Ct |
| Result | An ORF1ab | An ORF1ab |
| judgment | gene shows | gene shows |
| | positive | negative |
| | ROX fluorescence | |
| | | Ct > 40.0 or |
| Ct value | Ct ≤ 40.0 | no Ct |
| Result | An N gene shows | An N gene |
| judgment | positive | shows |
| | | negative |

When an ORF1ab gene shows positive or (and) an N gene shows positive, it can be judged that SARS-CoV-2 infection is positive, otherwise SARS-CoV-2 infection is negative.

In a third aspect, the present invention provides a kit for RT-PCR quantitative detection of Severe acute respiratory syndrome coronavirus 2 (SARS-CoV-2) with high sensitivity and specificity, which can accurately quantify a copy number of the SARS-CoV-2. In practice, the kit can not only perform early clinical diagnosis of people infected with the SARS-CoV-2, but also quantitatively detect treatment statuses of patients, and an effect is extremely good.

The present invention adopts a magnetic bead method to perform rapid nucleic acid lysis and adsorption on a sample, and adopts a fluorescence quantitative RT-PCR technology, that is, an initial template in a sample is quantitatively analyzed by monitoring fluorescence signals in a PCR system in real time. Meanwhile, optimal primer and probe sequences are designed, and a pseudotype virus containing COVID-19 and HCV gene fragments is used as a standard, and calibrated detection is conducted by HCV with an international standard, so that an effect of nucleic acid detection of the SARS-CoV-2 is further improved, and detection sensitivity is improved.

Due to influences of nucleic acid adsorption efficiency and collision efficiency of magnetic beads in a process of extracting an SARS-CoV-2 nucleic acid by the magnetic bead method, the magnetic bead method is more suitable for extraction of trace nucleic acids; an amount of a product can be detected in real time by quantitative RT-PCR, a standard curve is drawn by adding a known concentration of a pseudotype virus standard, and then a concentration of the initial template is calculated according to a position of a sample to be detected in the standard curve. It is easy and convenient to operate, provides accurate and reliable results, and is a new clinical detection method.

Therefore, in some implementations, the present invention provides use of a pseudotype virus standard containing SARS-CoV-2 and HCV gene fragments in preparation of a kit for quantitatively detecting a copy number of SARS-CoV-2.

Further, a gene of the pseudotype virus standard contains a target gene CoV/HCV RNA synthesized by connecting gene sequences of ORF1ab and N conserved regions of the SARS-CoV-2 and a conserved region of HCV in series, a sequence of the CoV/HCV RNA being shown in Seq ID NO. 10 in a sequence listing.

Experiments have proved that detection sensitivity can be significantly improved during nucleic acid extraction and detection of the SARS-CoV-2 by selecting sequence fragments of ORF1ab and N genes from the SARS-CoV-2 and designing appropriate primers and probes.

HCV conserved region genes are connected in series in the pseudotype virus standard, and a SARS-CoV-2 content of a pseudotype virus stock solution is subjected to calibrated detection by HCV with an international standard, so that a copy number of the standard is characterized very accurately.

Further, a preparation method of the pseudotype virus standard is as follows:
1) connecting gene sequences of an MS2 phage, the ORF1ab and N conserved regions of the SARS-CoV-2 and the conserved region of HCV in series and synthesizing a target gene MS2-CoV/HCV;
2) inserting the MS2-CoV/HCV gene into a prokaryotic expression vector pET-28b to construct a pET-28b-MS2-CoV/HCV recombinant plasmid; and
3) transforming the recombinant plasmid into E. coli competent cells, extracting a plasmid, conducting double enzyme digestion identification, conducting induced culture by IPTG, then identifying expression by SDS-PAGE, and conducting purification to obtain CoV/HCV RNA.

In particular, the gene sequence of the ORF1ab conserved region is shown in Seq ID NO.7, the gene sequence of the N conserved region is shown in Seq ID NO.8, the gene sequence of the conserved region of HCV is shown in Seq ID NO.9, and sequences of the MS2-CoV/HCV and the pET-28b-MS2-CoV/HCV are shown in Seq ID NO. 11 and Seq ID NO.12 in the sequence listing.

In another aspect, the present invention provides a kit for quantitatively detecting a copy number of SARS-CoV-2. The kit includes a pseudotype virus standard containing SARS-CoV-2 and HCV gene fragments.

In yet another aspect, the present invention provides a kit for quantitatively detecting a copy number of SARS-CoV-2. The kit includes two sets of primers and probes, and the two sets of primers and probes are respectively as follows.

A first set

A sequence of a forward primer corresponding to an ORF1ab gene as shown in Seq ID NO.1 in a sequence listing;

A sequence of a reverse primer corresponding to the ORF1ab gene as shown in Seq ID NO.2 in the sequence listing; and

A sequence of a probe corresponding to the ORF1ab gene as shown in Seq ID NO.3 in the sequence listing.

A second set

A sequence of a forward primer corresponding to an N gene as shown in Seq ID NO.4 in the sequence listing;

A sequence of a reverse primer corresponding to the N gene as shown in Seq ID NO.5 in the sequence listing; and

A sequence of a probe corresponding to the N gene as shown in Seq ID NO.6 in the sequence listing.

Further, the kit further includes a pseudotype virus standard containing SARS-CoV-2 and HCV gene fragments.

Further, a gene of the pseudotype virus standard contains a target gene CoV/HCV RNA synthesized by connecting gene sequences of ORF1ab and N conserved regions of the SARS-CoV-2 and a conserved region of HCV in series.

Further, a preparation method of the pseudotype virus standard is as follows:
1) connecting gene sequences of an MS2 phage, the ORF1ab and N conserved regions of the SARS-CoV-2 and the conserved region of HCV in series and synthesizing a target gene MS2-CoV/HCV;
2) inserting the MS2-CoV/HCV gene into a prokaryotic expression vector pET-28b to construct a pET-28b-MS2-CoV/HCV recombinant plasmid; and
3) transforming the recombinant plasmid into E. coli competent cells, extracting a plasmid, conducting double enzyme digestion identification, conducting induced culture by IPTG, then identifying expression by SDS-PAGE, and conducting purification to obtain CoV/HCV RNA.

Further, the kit further contains an HCV international standard.

A positive standard in the kit of the present invention is a phage-like pseudotype virus particle packaged with SARS-CoV-2 and HCV conserved gene fragments prepared by using an armored RNA technology. A SARS-CoV-2 content of a pseudotype virus stock solution is subjected to calibrated detection by HCV with an international standard, so that a copy number of the standard is characterized very accurately, which overcomes inaccuracy of relative judgment standards of a CT value by PCR analysis.

Further, the kit further includes a third set of internal standard primers and probes, which are as follows.

A sequence of a forward primer corresponding to a β-Globin gene as shown in Seq ID NO:13(5′-ttgcgtgatt atttagctga ctatgac -3′) in the sequence listing;

A sequence of a reverse primer corresponding to the β-Globin gene as shown in Seq ID NO:14(5′-tcctgtacca ccaacattaa tagca-3′) in the sequence listing; and

A sequence of a probe corresponding to the β-Globin gene as shown in Seq ID NO:15(5′- tcctgtacca ccaacattaa tagca-3′) in the sequence listing.

Further, the three sets of probes further include fluorescence generating genes and fluorescence quenching genes, a fluorescence generating gene of the probe corresponding to the ORF1ab gene being FAM, and a fluorescence quenching gene thereof being BHQ1; a fluorescence generating gene of the probe corresponding to the N gene being ROX, and a fluorescence quenching gene thereof being BHQ1; and a fluorescence generating gene of the probe corresponding to the β-Globin gene being VIC, and a fluorescence quenching gene thereof being BHQ1.

Further, the kit further includes a lysis solution, a washing solution, an eluent, a proteinase K, and magnetic beads.

A kit for quantitatively detecting a copy number of Severe acute respiratory syndrome coronavirus 2 (SARS-CoV-2) provided by the present invention includes a nucleic acid extraction kit and a nucleic acid amplification kit. The nucleic acid extraction kit includes a lysis solution, a washing solution, an eluent, a proteinase K, and magnetic beads.

The kit of the present invention adopts a magnetic bead method to extract viral nucleic acids for sample processing, and the lysis solution contains a strong protein denaturant, which rapidly dissolves a protein to release nucleic acids; released nucleic acid components can be bound to the magnetic beads; then, through the action of multiple washings, proteins, inorganic salt ions and various organic impurities are removed; and finally, purified nucleic acids are eluted with the eluent. After the technology is applied to the kit, on the one hand, the use of toxic reagents such as phenol, chloroform, and isoamyl alcohol in traditional methods is avoided, and on the other hand, mechanized operation is introduced, so that the influence of human operation factors on results is reduced.

Further, the lysis solution is a guanidine thiocyanate solution; the washing solution is an ethanol solution; and the eluent is a low-salt buffer or water.

Further, the lysis solution is a guanidine thiocyanate solution of 0.5 to 1 mol/L; the washing solution includes a washing solution I and a washing solution II, and the washing solution I is a 50% isopropanol solution, and the washing solution II is a 100% ethanol solution; the eluent is DEPC-treated water; a concentration of the proteinase K is 50 to 100 mol/L; and a solid content of the magnetic beads is 2.5%.

Further, Carrier RNA can also be added to the lysis solution. During nucleic acid extraction, if a concentration of an extracted product RNA needs to be increased, Carrier RNA (3 ul/sample, 1 ug/ul) can be added to the lysis solution.

Further, the kit further includes a reverse transcriptase, a DNA polymerase, dNTPs, a PCR buffer, a primer, and a probe.

The nucleic acid amplification kit includes an nCoV reaction solution, a positive standard, a positive quality control product and a negative quality control product. The nCoV reaction solution includes a reverse transcriptase, a DNA polymerase, dNTPs, a PCR buffer, a primer, and a probe.

Further, a usage amount of the reverse transcriptase is 50 to 200 U/reaction; a usage amount of the DNA polymerase is 1 to 8 U/reaction; the dNTPs are selected from a combination of dATP, dGTP, dCTP and dUTP, a combination ratio is 1:1:1:1 or 1:1:1:2, and a concentration is 10 to 25 mmol/L; a concentration of magnesium ions in the PCR buffer is 50 to 100 mmol/L; a concentration of the primer is 0.2 to 1 μmol/L; and a concentration of the probe is 0.05 to 0.1 μmol/L.

Further, the usage amount of the reverse transcriptase is 100 U/reaction; the usage amount of the DNA polymerase is 5 U/reaction; the dNTPs are selected from a combination of dATP, dGTP, dCTP and dUTP, a combination ratio is 1:1:1:2, and a concentration is 20 mmol/L; the concentration of the magnesium ions in the PCR buffer is 100 mmol/L; the concentration of the primer is 1 μmol/L; and the concentration of the probe is 0.1 μmol/L.

Further, the kit is characterized in that when the nucleic acid amplification kit performs nucleic acid amplification reaction, reaction temperatures and time are 50° C. for 20 minutes, 95° C. for 1 minute; reaction conditions are 95° C. for 3 seconds, 58° C. for 15 seconds, a total of 45 cycles; 37° C. for 10 seconds.

The present invention provides a kit for quantitatively detecting a copy number of Severe acute respiratory syndrome coronavirus 2 (SARS-CoV-2). The kit includes a nucleic acid extraction kit and a nucleic acid amplification kit. The nucleic acid extraction kit adopts a magnetic bead method to perform rapid nucleic acid lysis and adsorption on a sample. The nucleic acid amplification kit adopts a fluorescence quantitative RT-PCR technology for quantitative analysis. Meanwhile, optimal primer and probe sequences are designed, and a pseudotype virus containing SARS-CoV-2 and HCV gene fragments is used as a standard. A SARS-CoV-2 content of a pseudotype virus stock solution is subjected to calibrated detection by HCV with an international standard, so that a copy number of the standard is characterized very accurately, which overcomes inaccuracy of relative judgment standards of a CT value by fluorescent PCR analysis, further improves an effect of nucleic acid detection of the SARS-CoV-2, and improves detection sensitivity. The kit for quantitatively detecting the copy number of the SARS-CoV-2 provided by the present invention has relatively high sensitivity and specificity, can accurately quantify the copy number of the SARS-CoV-2, is easy and convenient to operate, and can provide accurate and reliable results.

Of course, it is also possible to directly perform amplification without lysing the samples for qualitative detection, which is convenient and quick.

### BRIEF DESCRIPTION OF DRAWINGS

FIG. 1 shows fluorescent PCR curves of positive quality control products of a kit of the present invention. In FIG. 1, 1 represents an amplification curve of an FAM channel of an SARS-CoV-2 positive quality control product, 2 represents an amplification curve of an ROX channel of the SARS-CoV-2 positive quality control product, and 3 represents an amplification curve of a VIC channel of an internal standard gene.

FIG. 2 shows a standard curve drawn according to a quantitative standard.

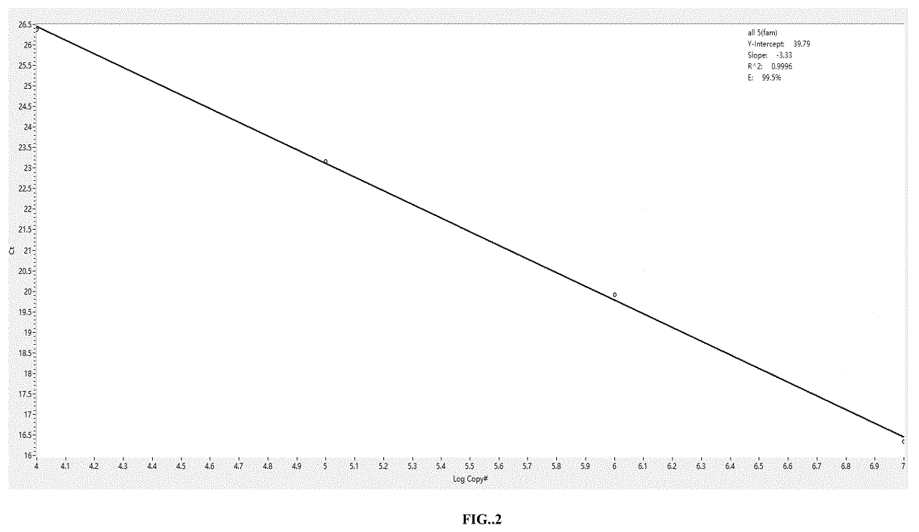

FIG. 3 shows a diagram of amplification curves of 4 pairs of primers corresponding to an ORF1ab gene of Embodiment 1.

FIG. 4 shows a diagram of amplification curves of 4 pairs of primers corresponding to an N gene of Embodiment 1.

### DETAILED DESCRIPTION OF EMBODIMENTS

The present invention has a kit and a detection method for detecting Severe acute respiratory syndrome coronavirus 2 (SARS-CoV-2), and adopts a Taqman probe method and a fluorescent PCR detection technology to rapidly detect a specific sequence of SARS-CoV-2 in clinical samples, so as to judge whether the SARS-CoV-2 exists in the samples. The present invention will be further described below in combination with specific embodiments, and the advantages and characteristics of the present invention will become more apparent with the description. These embodiments are only exemplary and do not constitute any limitation to the scope of the present invention. Test methods used in the following embodiments are all conventional methods unless otherwise specified. Materials, reagents, etc. used in the following embodiments can be obtained from commercial sources unless otherwise specified.

### Embodiment 1

### Development of Kit for Extraction-Free Fluorescent PCR Detection of SARS-CoV-2

### I. Design of Primers and Probes

Specific primers and probes were designed for an ORF1ab gene and an N gene by using a primer probe online design tool http://www.yeastgenome.org/cgi-bin/web-primer for the SARS-CoV-2 genome sequence published by the China National Center for Bioinformation/the National Genomics Data Center. Considering specificity and amplification efficiency, sequences of two sets of primer and probes finally selected were as follows:
sequences of ORF1ab gene-specific forward and reverse primers were respectively 5′-GGCTTCACATATGTATTGTTC-3′ and 5′-GCTCAAACTCTTCTTCTTCAC-3′; a sequence of an ORF1ab gene-specific probe was 5′-TCACCTTCTTCTTCATCCTCATCTGG-3′, and two ends of the probe were respectively labeled with a fluorescence generating group FAM and a fluorescence quenching group BHQ1; and
sequences of N gene-specific forward and reverse primers were respectively 5′-AAGGCTTCTACGCAGAAG-3′ and 5′-GCTGCCTGGAGTTGAATTTC-3′; a sequence of an N gene-specific probe was 5′-AGCCTCTTCTCGTTCCTCATCAC-3′, and two ends of the probe were respectively labeled with a fluorescence generating group ROX and a fluorescence quenching group BHQ2.

### II. Preparation of Target Gene Recombinant Plasmid

Shanghai Generay Biotech Co., Ltd. was entrusted to synthesize and purify a plasmid containing a SARS-CoV-2 target gene. A concentration was measured by an ultraviolet spectrophotometer, then a copy number was calculated, and diluting was conducted with 1×TE (pH 8.0) to a standard of 1.0×10⁵ copies/mL to 1.0×10² copies/mL respectively.

### III. Establishment and Optimization of Reaction System

Optimization of primer and probe concentrations: under a condition that other reaction components in a reaction system remained unchanged, primers with a concentration gradient of 0.2 μmol/L to 1 μmol/L and probes with a concentration gradient of 0.05 μmol/L to 0.1 μmol/L were used respectively for PCR reaction, and through multiple repeated experiments, an optimal primer concentration was finally determined to be 1 μmol/L, and a probe concentration was finally determined to be 0.1 μmol/L.

Optimization of a usage amount of a DNA polymerase: under a condition that other reaction components in a reaction system remained unchanged, an enzyme usage amount/reaction with a concentration gradient of 1 U to 5 U was used respectively, and through multiple repeated experiments, an optimal enzyme usage amount was finally determined to be 2 U/reaction.

Optimization of uracil-N-glycosylase (UNG): under a condition that other reaction components in a reaction system remained unchanged, an UNG usage amount/reaction with a concentration gradient of 0.1 U to 0.3 U was used respectively, and through multiple repeated experiments, an optimal UNG usage amount was finally determined to be 0.2 U/reaction.

Optimization of deoxy-ribonucleoside triphosphate: a deoxy-ribonucleoside triphosphate (dNTP) mixture was selected from a combination of dATP, dGTP, dCTP, and dUTP, and two ratios (1:1:1:1 and 1:1:1:2) were selected for comparison, results showed that the most preferred ratio is 1:1:1:2. dNTPs prepared according to the optimal ratio were used for multiple repeated experiments with a concentration gradient of 1 mmol/L to 3 mmol/L, and an optimal concentration was 2 mmol/L.

Optimization of a concentration of magnesium ions: under a condition that other reaction components in a reaction system remained unchanged, magnesium ions with a concentration gradient of 50 mmol/L to 100 mmol/L were used respectively, and through multiple repeated experiments, an optimal concentration of the magnesium ions was finally determined to be 100 mmol/L.

Optimization of a reaction temperature and time: according to activity of enzymes and lengths of oligonucleotide and polynucleotide, the reaction temperature and extension time were mainly optimized, and an optimal reaction temperature and time were finally determined to be 50° C. for 15 minutes and 96° C. for 60 seconds; PCR reaction conditions were 96° C. for 3 seconds, 58° C. for 15 seconds, a total of 45 cycles.

### IV. Detection Results

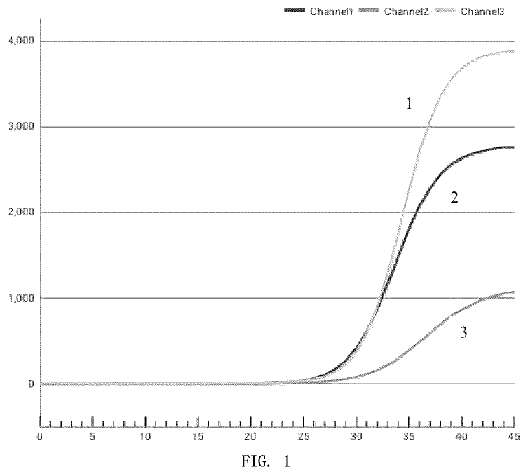

Positive quality control products of the kit were a pUC57 vector plasmid containing an SARS-CoV-2-specific conserved target sequence and a vector plasmid of a target fragment amplified by an internal standard primer. Experimental data showed that the positive quality control products and an internal standard solution of the kit have excellent amplification effects. Referring to FIG. 1 for experimental results.

### Embodiment 2

### Detection of Actual Clinical Samples

1. Types of clinical samples. The clinical samples used in this embodiment came from throat swab samples collected by the Zhejiang Provincial Centre For Disease Control And Prevention. Collected swabs were stored in normal saline or a virus preservation solution, with a total of 301 cases, including 105 cases of positive samples and 106 cases of negative samples.
2. Reference kits. Kits used by the Zhejiang Provincial Centre for Disease Control and Prevention were used as reference reagents.
3. Detection Results

301 cases were selected for detection, of which 2 cases were rejected because samples thereof were thick sputum, which was inconsistent with a sample type required by the kits, and 4 cases were rejected because internal standards were not available and detection results were ineffective, with a total of 295 effective cases. All effective results were included in statistical analysis (Table 2).

| Kits of the | | | |
|---|---|---|---|
| present | Reference kits | | |
| invention | Positive | Negative | Total |
| Positive | 99 | 1 | 100 |
| Negative | 6 | 89 | 195 |
| Total | 105 | 190 | 295 |

### Result Analysis

Statistics of results of the kits of the present invention and results of the reference kits shows that a positive coincidence rate is 94.29% (87.98% to 97.87%), a negative coincidence rate is 99.47% (97.10% to 99.99%), and a total coincidence rate is 97.63% (95.17% to 99.04%).

### Embodiment 3

### Analysis Description of Mutant Virus Strains by Using Primers of the Present Invention

SARS-CoV-2 mutation monitoring report:
1. SARS-CoV-2 D614G mutation refers to a mutant strain in which an amino acid on a 614th position on an S protein is mutated from D to G. The mutation was produced in Europe before Jan. 15, 2020. Before March 21, the 614th position of the S protein of a SARS-CoV-2 strain that was epidemic in the world was mainly D, and after that, the mutant strain whose 614th position was G became a main epidemic strain. A vast majority of viruses currently epidemic in the world are virus strains with the D614G mutation.
2. SARS-CoV-2 strain B.1.1.7 was first found in the UK, with 17 new mutation sites, 8 mutations occurred in an S protein region, 2 mutations occurred in an N protein region, 4 mutations occurred in an ORF1ab segment, and there were also two mutations in other regions. Among these mutations, an N501Y mutation of the S protein belongs to a receptor binding motif. This mutation can increase an affinity of SARS-CoV-2 with a human ACE2 receptor and enhance an ability of the virus to enter cells, so that the transmissibility of the SARS-CoV-2 can be increased by 70%, while a P681 mutation and a 69-70del mutation may be related to immune evasion of the SARS-CoV-2. At present, the virus strain has been found and epidemic in 31 countries around the world.
3. SARS-CoV-2 mutant strain B.1.351 (501Y V2) was first found in South Africa. In addition to N501Y and D614G, its main mutation sites also include two S protein key mutations K417N and E484K. In addition, B.1.351 has an N protein T2051 mutation and an ORF1a mutation K1655N. The mutant strain may be epidemic in the youth population. At present, the mutant strain has been found in 8 regions around the world. 4. Brazilian SARS-CoV-2 mutant strain P.1 (B.1.1.28.1) contains 16 mutations, and key mutations thereof are S protein K417N, E484K and N501Y. Other mutations also include ORF1ab S1188L and K1795Q. The transmissibility of the mutant strain was also enhanced. At present, 6 districts around the world have found that the virus is spreading. Compared with the aforementioned three SARS-CoV-2 mutant strains, it has not yet been found in China.

Table 3 Summary table of three major SARS-CoV-2 mutant strains at present

| SARS- | | | |
|---|---|---|---|
| CoV-2 | | | |
| mutant | First appeared | | |
| strains | District | Date | Mutation (nucleic acid change) |
| B.1.1.7 | UK | September | ORF1ab: T1001I, A1708D, I2230T, del3675-3677 |
| (501Y.V1) | | 2020 | SGF |
| | | | S: del69-70 HV, del144 Y, N501Y, A570D, D614G, |
| | | | P681H, T761I, S982A, D1118H |
| | | | ORF8: Q27stop, R52I, Y73C |
| | | | N: D3L, S235F |
| B.1.351 | South | October | ORF1ab: K1655N |
| (501Y.V2) | Africa | 2020 | S: K417N, E484K, N501Y, D614G, A701V, |
| | | | E: P71L |
| | | | N: T205I |
| P.1 | Brazil/ | January 2021 | ORF1ab: F681L, I760T, S1188L, K1795Q, |
| (501Y.V3) | Japan | | del3675-3677 SGF, E5662D |
| | | | S: L18F, T20N, P26S, D138Y, R190S, K417T, |
| | | | E484K, N501Y, D614G, H655Y, T1027I |
| | | | ORF3a: C174G |
| | | | ORF8: E92K |
| | | | ORF9: Q77E |
| | | | ORF14: V49L |
| | | | N: P80R |

Note:
underlined red bold fonts represent characteristic mutations.

The above reagents of the present invention used a nucleic acid release technology to release viral nucleic acids, and used a fluorescent PCR method for nucleic acid detection. This product compared the similarities and differences of an ORF1ab gene and an N gene among various coronavirus strains, and designed specific primers and probes accordingly, and meanwhile used a human house-keeping gene β-Globin gene sequence as a template to design primers and probes to serve as internal control indicators, among which 2019-nCoV specific probes were labeled with FAM fluorescence and ROX fluorescence respectively, and an internal standard gene was labeled with VIC fluorescence. On a fluorescence quantitative PCR instrument, the detection of 2019-nCoV RNA of samples was realized through the change of fluorescent signals.

This product detects 2019-nCoV sequences (Genbank No: NC_045512) nt3012-nt3082 (ORF1ab region) and nt28778-nt28875 (N gene), a total of 2 segments. Through sequence comparison and analysis, gene mutation ranges of the mutant SARS-CoV-2 strain B.1.1.7 first found in the UK, the SARS-CoV-2 mutant strain B.1.351 (501Y V2) first found in South Africa, and the SARS-CoV-2 mutant strain P.1 found in Brazil were all not in a detection segment of the kit, so the kit can be used for the detection of these three mutant viruses. Although it cannot distinguish which mutants are, it can cover tests of these three mutant viruses. For detailed analysis and comparison summary, see Table 3.

Table 4 Comparison table of SARS-CoV-2 nucleic acid detection segments of the present invention and epidemic mutant SARS-CoV-2 segments

| SARS-CoV- | | | Base mutation positions |
|---|---|---|---|
| 2 | | | related to detection of |
| mutant | First appeared | | amplified fragments (segments) |
| strains | District | Date | of the present invention |
| SARS- | ORF1ab | | nt3012-nt3082 |
| CoV-2 nucleic | N gene | | nt28778-nt28875 |
| acid | | | |
| detection | | | |
| segments | | | |
| of the | | | |
| present | | | |
| invention | | | |
| B.1.1.7 | UK | September | ORF1ab: C3267T, C5388A, T6954C, |
| (501Y.V1) | | 2020 | 11288-11296del |
| | | | N: 28280 GAT > CTA, C28977T |
| B.1.351 | South | October | ORF1ab: C1059T, G5230T, |
| (501Y.V2) | Africa | 2020 | C8660T, C8964T, A10323G, |
| | | | G13843T, C14408T, C17999T |
| | | | N: none |
| P.1 | Brazil/ | January | ORF1ab: 11288-11296del |
| (501Y.V3) | Japan | 2021 | N: C28887T |

Note:
(1) a sequence is an NCBI SARS-CoV-2 standard sequence (NC_045512);
(2) SARS-CoV-2 nucleic acid detection reagents of the present invention refer to the kits described in Embodiment 1 and the kits described in Embodiment 2.

### Embodiment 4

### Component Preparation of Kit for Quantitatively Detecting Copy Number of SARS-CoV-2 and Detection Method

### I. Experimental Equipment, Materials and Instruments

1. Fluorescent genes: VIC: green fluorescent protein; FAM: 6-Carboxy-fluorescein; HEX: 5-Hexachloro-flurescein; Cy5: Indodicarbocyanine (Cy5 flurescein); Rox: Carboxy-x-rhodamine; TET: 5-Tetrachloro-fluorescein.
2. Fluorescence quenching genes: BHQ-1: BlackHole Quencher-1; BHQ-2: Black Hole Quencher-2; BHQ-3: BlackHole Quencher-3; BBQ: BlackBerry Quencher; TAMRA: Tetramethyl-6-carboxyrhodamine.

In the present invention, the above-mentioned fluorescent dyes (fluorescent genes) and the fluorescence quenchers (fluorescence quenching genes) are only partial examples, and other reagents can also be used. In the various embodiments of the present invention, the fluorescent genes, the fluorescence quenching genes, enzymes, glycerol, and nucleic acid-free water were all conventionally purchased from the market, specifically, they might be quantitative reaction solutions, etc. purchased from Thermo Fisher Life, Roche. Of course, they were not limited to the above-mentioned companies, and might also be purchased from companies such as ABI, BIO-RAD, etc. in the United States.
3. Control products: a positive standard was pseudotype virus CoV/HCV RNA, and an HCV international standard used for calibration was purchased from Shenzhen Yuanyang Biotechnology Co., Ltd.; a positive quality control product was prepared with a TE Buffer and the pseudotype virus CoV/HCV RNA according to required concentrations; a negative quality control product was a TE Buffer containing an internal standard, and a storage condition thereof was below −20±10° C.
4. Equipment: Allsheng Auto-Pure32A Magnetic Bead Nucleic Acid Extractor and similar instruments were used for nucleic acid extraction; ABI 7500 amplification instrument was used for RT-PCR.

### II. Component Preparation of Kit for Quantitatively Detecting Copy Number of SARS-CoV-2 and Detection Method

### 1. Nucleic Acid Extraction

1) Kit components: a lysis solution being a guanidine thiocyanate solution of 0.5 to 1 mol/L, a washing solution I being a 50% isopropanol solution, a washing solution II being a 100% ethanol solution, an eluent being DEPC-treated water, a proteinase K of 50 to 100 mol/L, and magnetic beads with a solid content of 25%. In particular, the lysis solution, the washing solution I and the washing solution II were subpackaged in different bottles, which are called a lysis solution bottle, a washing solution I bottle, and a washing solution II bottle.
2) When using for the first time, isopropanol or absolute ethanol was added to the above bottles separately, in particular, 15 mL of absolute ethanol was added to the lysis solution bottle; 40 mL of absolute ethanol was added to the washing solution II bottle. After even mixing, markers were done on bottle bodies and the bottles were preserved in a sealed mode at a room temperature.
3) According to the number of samples (two samples can be processed in one horizontal row), as shown in Table 4, the following reagents and samples were added to corresponding slots of a 96-well plate:

TABLE 4
Reagent additions in corresponding slots in 96-well plate

| Well/ | | |
|---|---|---|
| column | Reagent | Volume |
| 1/7 | Lysis solution | 600 μl |
| 1/7 | Magnetic | 20 μl |
| | beads | |
| 1/7 | Proteinase K | 25 μl |
| 2/8 | Washing | 500 μl |
| | solution I | |
| 3/9 | Washing | 500 μl |
| | solution II | |
| 4/10 | Washing | 500 μl |
| | solution II | |
| 5/11 | Null | / |
| 6/12 | Eluent | 60 μl |

If a concentration of an extracted product RNA needs to be increased, Carrier RNA (3 ul/sample, 1 ug/ul) can be added to a lysis solution well.
5) 400 μl of a sample to be processed was added to wells of 1st and 7th columns, operating parameters were set as shown in Table 5 and operated.

TABLE 5
Operating parameter settings

| | | | | | Magnetic | | | | | | |
|---|---|---|---|---|---|---|---|---|---|---|---|
| | | | Waiting | Mixing | attraction | Magnetic | | Even | | | |
| | | | time | time | time | attraction | Volume | mixing | Oscillation | Temperature | Temperature |
| Step | Slot | Name | (min) | (min) | (sec) | position | (μl) | speed | amplitude | status | (° C.) |
| 1 | 1/7 | Lysis/ | 0 | 15 | 90 | 0% | 940 | 6 | 60% | Heated | 85 |
| | | binding | | | | | | | | | |
| 2 | 2/8 | Washing 1 | 0 | 2 | 60 | 0% | 500 | 4 | 60% | None | — |
| 3 | 3/9 | Washing 2 | 0 | 1 | 60 | 0% | 500 | 4 | 60% | None | — |
| 4 | 4/10 | Washing 3 | 0 | 0 | 60 | 0% | 500 | 4 | 60% | None | |
| 5 | 5/11 | Air-drying | 1 | 0 | | 100% | 500 | 1 | — | None | — |
| 6 | 6/12 | Elution | 0 | 4 | 120 | 0% | 100 | 5 | 60% | Heated | 65 |
| 7 | 2/8 | Discarding | 0 | 1 | 0 | 0% | 500 | 5 | 60% | None | — |
| | | magnetic | | | | | | | | | |
| | | beads | | | | | | | | | |

6) After instruments finished running, the 96-well plate was removed.
7) Eluted products in 6th and 12th columns were transferred to clean centrifuge tubes for next experiment or being stored at −20±5° C.

### 2. PCR Amplification and Detection

1) Design of primers and probes: specific primers and probes were designed for an ORF lab gene and an N gene by using a primer probe online design tool http://www.yeastgenome.org/cgi-bin/web-primer for the SARS-CoV-2 genome sequence published by the China National Center for Bioinformation/the National Genomics Data Center.

Considering specificity and amplification efficiency, sequences of two sets of primer and probes finally selected were as follows:
sequences of ORF1ab gene-specific forward and reverse primers were respectively 5′-TTTCCACTGTTTTCGTGCC-3′ and 5′-GACCCATAGTAGCGCAGAG-3′; a sequence of an ORF1ab gene-specific probe was 5′-CAGTTCCTCTTCACATAATCGCCCCGAG-3′, and two ends of the probe were respectively bound with a fluorescence generating group FAM and a fluorescence quenching group BHQ1; and
sequences of N gene-specific forward and reverse primers were respectively 5′-GTGCTGTTTCCTTTGCCGATA-3′ and 5′-CAGTTCCTCTTCACATAATCGCCCCGAG-3′; a sequence of an N gene-specific probe was 5′-ACAGTGTTATTTGGTGCAGCTCGTGGTT-3′, and two ends of the probe were respectively bound with a fluorescence generating group ROX and a fluorescence quenching group BHQ1.

### 2) Establishment and Optimization of Reaction System

Optimization of primer and probe concentrations: under a condition that other reaction components in a reaction system remained unchanged, primers with a concentration gradient of 0.2 μmol/L to 1 μmol/L and probes with a concentration gradient of 0.05 μmol/L to 0.1 μmol/L were used respectively for PCR reaction, and through multiple repeated experiments, an optimal primer concentration was finally determined to be 1 μmol/L, and a probe concentration was finally determined to be 0.1 μmol/L.

Optimization of a usage amount of a DNA polymerase: under a condition that other reaction components in a reaction system remained unchanged, an enzyme usage amount/reaction with a concentration gradient of 1 U to 8 U was used respectively, and through multiple repeated experiments, an optimal enzyme usage amount was finally determined to be 5 U/reaction.

Optimization of a usage amount of an RT enzyme: under a condition that other reaction components in a reaction system remained unchanged, an enzyme usage amount/reaction with a concentration gradient from 50 U (enzyme usage amount) to 200 U was used respectively for RT-PCR reaction, and through multiple repeated experiments, an optimal RT enzyme usage amount was finally determined to be 100 U/reaction.

Optimization of deoxy-ribonucleoside triphosphate: a deoxy-ribonucleoside triphosphate (dNTP) mixture was selected from a combination of dATP, dGTP, dCTP, and dUTP, and two ratios (1:1:1:1 and 1:1:1:2) were selected for comparison, results showed that the most preferred ratio is 1:1:1:2. dNTPs prepared according to the optimal ratio were used for multiple repeated experiments with a concentration gradient of 10 mmol/L to 25 mmol/L, and an optimal concentration was 20 mmol/L.

Optimization of a concentration of magnesium ions: under a condition that other reaction components in a reaction system remained unchanged, magnesium ions with a concentration gradient of 50 mmol/L to 100 mmol/L were used respectively, and through multiple repeated experiments, an optimal concentration of the magnesium ions was finally determined to be 100 mmol/L.

Optimization of a reaction temperature and time: according to activity of enzymes and lengths of oligonucleotide and polynucleotide, the reaction temperature and extension time were mainly optimized, and an optimal reaction temperature and time were finally determined to be 50° C. for 20 minutes and 95° C. for 1 minute; PCR reaction conditions were 95° C. for 3 seconds, 58° C. for 15 seconds, a total of 45 cycles; 37° C. for 10 seconds.

According to optimization results, optimal configuration of a reaction system of a nucleic acid amplification kit was determined as shown in Table 6.

A usage amount of a reverse transcriptase is 100 U/reaction; the usage amount of the DNA polymerase is 5 U/reaction; the dNTPs are selected from a combination of dATP, dGTP, dCTP and dUTP, a combination ratio is 1:1:1:2, and a concentration is 20 mmol/L; the concentration of the magnesium ions in the PCR buffer is 100 mmol/L; the concentration of the primer is 1 μmol/L; and the concentration of the probe is 0.1 μmol/L.

TABLE 6
Optimal configuration of reaction system
of nucleic acid amplification kit

| | | Volume/ |
|---|---|---|
| Components | Detailed compositions | reaction (ul) |
| Enzyme | Reverse transcriptase of 100 | 5 |
| | U/reaction; | |
| | DNA polymerase of 5 | |
| | U/reaction | |
| PCR buffer | A concentration of magnesium | 25 |
| | ions in the PCR buffer was | |
| | 100 mmol/L; a combination | |
| | ratio of dATP, dGTP, dCTP, | |
| | and dUTP was 1:1:1:2, and a | |
| | concentration was 20 mmol/L | |
| ORF1ab | A primer concentration was | 0.1 |
| upstream primer | 0.2 μmol/L | |
| ORF1ab | A primer concentration was | 0.1 |
| downstream | 0.2 μmol/L | |
| primer | | |
| ORF1ab probe | A probe concentration was 0.1 | 0.05 |
| | μmol/L | |
| N upstream | A primer concentration was | 0.1 |
| primer | 0.2 μmol/L | |
| N downstream | A primer concentration was | 0.1 |
| primer | 0.2 μmol/L | |
| N probe | A probe concentration was 0.1 | 0.05 |
| | μmol/L | |
| β-Globin | A primer concentration was | 0.1 |
| upstream primer | 0.2 μmol/L | |
| β-Globin | A primer concentration was | 0.1 |
| downstream | 0.2 μmol/L | |
| primer | | |
| β-Globin probe | A probe concentration was 0.1 | 0.05 |
| | μmol/L | |

### 3) Reading of Copy Numbers of Positive Standard and Detection Samples

A kit was configured according to Tables 1, 2 and 3 respectively. A positive standard in the kit is pseudotype virus CoV/HCV RNA with a concentration of 10² to 10⁷ copies/ml. A fluorescent PCR instrument will draw a standard curve according to a quantitative standard (as shown in FIG. 1), and an SARS-CoV-2 content in detection samples was determined accordingly.

### Embodiment 5

### Design of Primer-Probe Sets in Kits for Quantitatively Detecting Copy Number of SARS-CoV-2

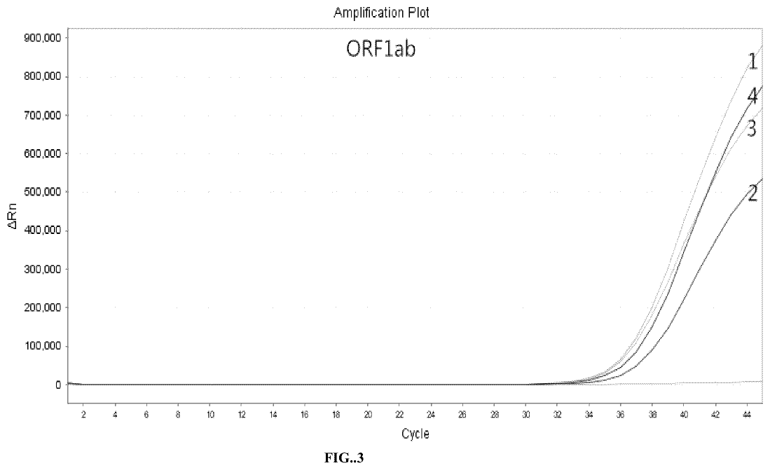

In this embodiment, multiple pairs of specific primers and probes were designed for an ORF1ab gene and an N gene of SARS-CoV-2, respectively and compared. A large number of experiments have proved that different primers have a certain influence on an effect of PCR amplification and detection sensitivity of kits. In this embodiment, four sets of primer-probes were designed for the ORF1ab gene and the N gene, respectively, including ORF1ab-1, ORF1ab-2, ORF1ab-3 and ORF1ab-4 corresponding to the ORF1ab gene (primer sequences were shown in Table 4); N-1, N-2, N-3 and N-4 corresponding to the N gene (primer sequences were shown in Table 5). These sequences could specifically bind to corresponding sequences of the ORF1ab gene and the N gene of the SARS-CoV-2, respectively. Amplification curves of positive samples of the ORF1ab gene were shown in FIG. 3, and amplification curves of positive samples of the N gene were shown in FIG. 7.

TABLE 7
Sequence information of ORF1ab-1, ORF1ab-2,
ORF1ab-3 and ORF1ab-4

| Set | Name | Sequence |
|---|---|---|
| ORF1ab- | Forward | TTTCCACTGTTTTCGTGCC (SEQ IN NO: 1) |
| 1 | Reverse | GACCCATAGTAGCGCAGAG (SEQ IN NO: 2) |
| | Probe | CAGTTCCTCTTCACATAATCGCCCCGAG (SEQ |
| | | IN NO: 3) |
| ORF1ab- | Forward | ACTGGACTTTATTGACACTA (SEQ IN NO: |
| 2 | | 16) |
| | Reverse | CGTGTACCAAGCAATTTC (SEQ IN NO: 17) |
| | Probe | ATACTGCTGCCGTGAA (SEQ IN NO: 18) |
| ORF1ab- | Forward | GGATCAAGAATCCTTTGG (SEQ IN NO: 19) |
| 3 | Reverse | AGCACAAGTTGTAGGTA (SEQ IN NO: 20) |
| | Probe | ACTGCCGTTGCCACATAGAT (SEQ IN NO; |
| | | 21) |
| ORF1ab- | Forward | CGTCTACAAGCTGGTA (SEQ IN NO: 22) |
| 4 | Reverse | TTCCGGTGTAACTGTTA (SEQ IN NO: 23) |
| | Probe | TGCCTGCCAATTCAACTGTATTATC (SEQ IN |
| | | NO: 24) |

TABLE 8
Sequence information of N-1, N-2, N-3 and N-4

| Set | Name | Sequence |
|---|---|---|
| N-1 | Forward | GTGCTGTTTCCTTTGCCGATA (SEQ IN |
| | | NO; 4) |
| | Reverse | CAGTTCCTCTTCACATAATCGCCCCGAG (SEQ |
| | | IN NO: 5) |
| | Probe | ACAGTGTTATTTGGTGCAGCTCGTGGTT (SEQ |
| | | IN NO: 6) |
| N-2 | Forward | ACTCCAGGCAGCAGTAGG (SEQ IN NO: 25) |
| | Reverse | AGCAGCAGCAAAGCAAGA (SEQ IN NO: 26) |
| | Probe | CATCACCGCCATTGCCAGCCATTC (SEQ IN |
| | | NO: 27) |
| N-3 | Forward | AGGCAGCAGTAGGGGAAC (SEQ IN NO; 28) |
| | Reverse | CAAGCAGCAAAGCAA (SEQ IN NO: 29) |
| | Probe | CATCACCGCCATTGCCAGCCATTC (SEQ IN |
| | | NO: 30) |
| N-4 | Forward | GCTGTTTCCTTTGCCGATA (SEQ IN NO: 31) |
| | Reverse | AGCAGCAGCAAAGCAA (SEQ IN NO: 32) |
| | Probe | AGTGTTATTTGGTGCAGCTCGTGG (SEQ IN |
| | | NO: 33) |

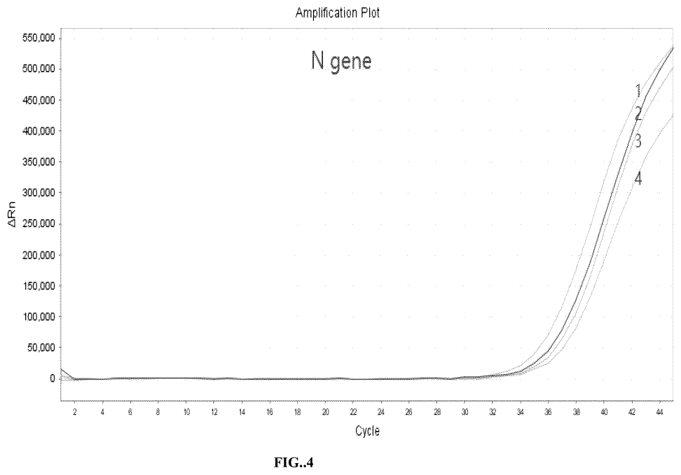

It can be seen from FIG. 3 and FIG. 4 that the amplification curves of ORF1ab-1 and N-1 are the best, with the earliest peak appearance and the highest fluorescence intensity, and have obvious exponential phases and plateau phases. Other primer-probe curves have lower rising peak heights and later peak appearance time, which are weaker than ORF1ab-1 and N-1, respectively. It shows that target products of primers and probes of ORF1ab-1 and N-1 have higher replication speeds, more quantities and higher amplification reaction efficiency.

Kits were configured according to Table 1, Table 2 and Table 3. A kit configured with ORF1ab-1 and N-1 was designated as Kit 1, and a kit configured with ORF1ab-2 and N-2 was designated as Kit 2, they both used primers and probes corresponding to the β-Globin gene shown in Seq ID NO.10, Seq ID NO.11, and Seq ID NO.12 in a sequence listing as internal standard primers and probes. The detection sensitivity of Kit 1 and the detection sensitivity of Kit 2 were compared. Pseudotype virus CoV/HCV RNA as a positive control was selected as samples. Sample solutions of 10 to 10⁶ copies were prepared according to template concentrations shown in Table 6 and Table 7, and were calibrated with HCV. The samples at each concentration were detected three times. Detection results were shown in the Table 6 and Table 7.

TABLE 6
Detection results of Kit 1

| Template concentration | | Ct value-(internal |
|---|---|---|
| (added template) | Ct value-(sample) | standard) |
| 10⁶ copies (ORF1ab-1) | 21.51 | 17.13 |
| | 21.20 | 17.22 |
| | 21.17 | 17.15 |
| 10⁵ copies (ORF1ab-1) | 23.68 | 16.73 |
| | 23.76 | 16.88 |
| | 23.59 | 16.92 |
| 10⁴ copies (ORF1ab-1) | 27.09 | 17.18 |
| | 26.83 | 17.07 |
| | 27.10 | 16.96 |
| 10³ copies (ORF1ab-1) | 31.35 | 17.01 |
| | 31.41 | 17.14 |
| | 31.23 | 17.27 |
| 10² copies (ORF1ab-1) | 37.15 | 17.32 |
| | 37.21 | 17.13 |
| | 37.17 | 17.24 |
| 10 copies (ORF1ab-1) | Undetermined | 17.17 |
| | Undetermined | 17.23 |
| | Undetermined | 17.29 |
| Negative control | Undetermined | 18.01 |
| | Undetermined | 17.81 |
| 10⁶ copies (N-1) | 20.13 | 16.87 |
| | 19.95 | 16.99 |
| | 20.15 | 16.58 |
| 10⁵ copies (N-1) | 22.32 | 16.76 |
| | 22.44 | 16.58 |
| | 22.81 | 16.63 |
| 10⁴ copies (N-1) | 25.53 | 17.26 |
| | 25.71 | 17.17 |
| | 25.68 | 17.04 |
| 10³ copies (N-1) | 30.75 | 16.94 |
| | 30.54 | 16.76 |
| | 30.82 | 16.98 |
| 10² copies (N-1) | 36.97 | 17.04 |
| | 36.81 | 17.23 |
| | 36.85 | 17.17 |
| 10 copies (N-1) | Undetermined | 17.03 |
| | Undetermined | 17.18 |
| | Undetermined | 17.27 |
| Negative control | Undetermined | 17.51 |
| | Undetermined | 17.86 |

TABLE 7
Detection results of Kit 2

| Template concentration | | |
|---|---|---|
| (added template) | Ct value-(sample) | Ctvalue-(internal standard) |
| 10⁶ copies (ORF1ab-2) | 22.04 | 19.61 |
| | 22.15 | 19.82 |
| | 21.79 | 19.73 |
| 10⁵ copies (ORF1ab-2) | 26.18 | 20.17 |
| | 26.57 | 19.24 |
| | 26.72 | 20.21 |
| 10⁴ copies (ORF1ab-2) | 31.41 | 20.17 |
| | 31.15 | 20.24 |
| | 31.44 | 19.83 |
| 10³ copies (ORF1ab-2) | 36.13 | 20.04 |
| | 36.02 | 20.01 |
| | 36.07 | 19.83 |
| 10² copies (ORF1ab-2) | Undetermined | 20.05 |
| | Undetermined | 19.67 |
| | Undetermined | 19.66 |
| 10 copies (ORF1ab-2) | Undetermined | 20.17 |
| | Undetermined | 20.21 |
| | Undetermined | 20.25 |
| Negative control | Undetermined | 20.87 |
| | Undetermined | 21.07 |
| 10⁶ copies (N-2) | 20.13 | 20.17 |
| | 21.15 | 20.13 |
| | 21.07 | 20.01 |
| 10⁵ copies (N-2) | 25.14 | 19.97 |
| | 25.11 | 20.05 |
| | 25.20 | 20.03 |
| 10⁴ copies (N-2) | 30.85 | 20.17 |
| | 31.32 | 20.18 |
| | 31.07 | 20.08 |
| 10³ copies (N-2) | 36.16 | 20.17 |
| | 36.71 | 19.91 |
| | 35.88 | 20.17 |
| 10² copies (N-2) | Undetermined | 20.15 |
| | Undetermined | 20.18 |
| | Undetermined | 20.20 |
| 10 copies (N-2) | Undetermined | 20.27 |
| | Undetermined | 20.05 |
| | Undetermined | 20.27 |
| Negative control | Undetermined | 20.74 |
| | Undetermined | 21.11 |

According to Table 6, it can be seen from that when Kit 1 is used for detection, a detection limit of ORF1ab is 10² copies, a Ct value thereof is 37.15 to 37.21, a detection limit of N is 102 copies, and a Ct value thereof is 36.81 to 36.97; according to Table 7, it can be seen that when Kit 2 is used for detection, a detection limit of ORF1ab is 103 copies, a Ct value thereof is 36.07 to 36.31, and a detection limit of N is 103 copies, and a Ct value thereof is 35.88 to 36.71. It can be seen that the detection limit of Kit 1 is lower, a content of a lower concentration virus can be detected, and the detection sensitivity is significantly higher than that of Kit 2, thus it is proved that the detection sensitivity of kits for quantitatively detecting a copy number of SARS-CoV-2 including primers and probes designed corresponding to ORF1ab-1 and N-1 is higher.

### Embodiment 6

### Preparation and Calibration of Pseudotype Virus Standard

This embodiment used an armored RNA technology to prepare a pseudotype virus packaged with SARS-CoV-2 and HCV gene fragments to be used as a positive standard in a kit of the present invention. A SARS-CoV-2 content of a pseudotype virus stock solution was subjected to calibrated detection by HCV with an international standard, so that a copy number of the standard was characterized very accurately, which overcame inaccuracy of relative judgment standards of a CT value by PCR analysis, and further improved sensitivity of nucleic acid detection of the SARS-CoV-2.

In this embodiment, according to the characteristics of the SARS-CoV-2, ORF1ab and N conserved regions (a gene sequence of the ORF1ab conserved region was shown in Seq ID NO:7(5′-ggcttcacat atgtattgtt ctttctaccc tccagatgag gatgaagaag aaggtgattg tgaagaagaa gagtttgagc-3′), and a gene sequence of the N conserved region was shown in Seq ID NO:8(5′-ggcttcacat atgtattgtt ctttctaccc tccagatgag gatgaagaag aaggtgattg tgaagaagaa gagtttgagc-3′) were selected for packaging of the pseudotype virus, these two segments well characterized characteristic sequences of the SARS-CoV-2, and also connected a conserved region of HCV as shown in Seq ID NO:9(5′-atggtagctg gcacataaac cggaccgctc tcaattgcaa tgacagcttg caaacgggtt tcctcgcctc cctgttttac acccacagct tcaacagctc tggctgcccc gagcgcttgt cttcctgccg-′3) and an MS2 phage in series and synthesized a target gene MS2-CoV/HCV (Seq ID NO.11) as shown below sequence:.

(Seq ID NO. 11)
tggctatcgc tgtaggtagc cggaattcca ttcctaggag
gtttgacctg tgcgagcttttagtaccctt gatagggaga
acgagacctt cgtcccctcc gttcgcgttt acgcggacgg
tgagactgaa gataactcat tctctttaaa atatcgttcg
aactggactc ccggtcgttt taactcgact ggggccaaaa
cgaaacagtg gcactacccc tctccgtatt cacggggggc
gttaagtgtc acatcgatag atcaaggtgc ctacaagcga
agtgggtcat cgtggggtcg cccgtacgag gagaaagccg
gtttcggctt ctccctcgac gcacgctcct
gctacagcctcttccctgta agccaaaact tgacttacat
cgaagtgccg cagaacgttg cgaaccgggc gtcgaccgaa
gtcctgcaaa aggtcaccca gggtaatttt aaccttggtg
ttgctttagc agaggccagg tcgacagcct cacaactcgc
gacgcaaacc attgcgctcg tgaaggcgta cactgccgct
cgtcgcggta attggcgcca ggcgctccgc taccttgccc
taaacgaaga tcgaaagttt cgatcaaaac acgtggccgg
caggtggttg gagttgcagt tcggttggtt accactaatg
agtgatatcc agggtgcata tgagatgctt acgaaggttc
accttcaaga gtttcttcct atgagagccg tacgtcaggt
cggtactaac atcaagttag atggccgtct gtcgtatcca
gctgcaaact tccagacaac gtgcaacata tcgcgacgta
tcgtgatatg gttttacata aacgatgcac gtttggcatg
gttgtcgtct ctaggtatct tgaacccact aggtatagtg
tgggaaaagg tgcctttctc attcgttgtc gactggctcc
tacctgtagg taacatgctc gagggcctta cggcccccgt
gggatgctcc tacatgtcag gaacagttac tgacgtaata
acgggtgagt ccatcataag cgttgacgct ccctacgggt
ggactgtgga gagacagggc actgctaagg cccaaatctc
agccatgcat cgaggggtac aatccgtatg gccaacaact
ggcgcgtacg taaagtctcc tttctcgatg gtccatacct
tagatgcgtt agcattaatc aggcaacggc tctctagata
gagccctcaa ccggagtttg aagcatggct tctaacttta
ctcagttcgt tctcgtcgac aatggcggaa ctggcgacgt
gactgtcgcc ccaagcaact tcgctaacgg ggtcgctgaa
tggatcagct ctaactcgcg ttcacaggct tacaaagtaa
cctgtagcgt tcgtcagagc tctgcgcaga atcgcaaata
caccatcaaa gtcgaggtgc ctaaagtggc aacccagact
gttggtggtg tagagcttcc tgtagccgca tggcgttcgt
acttaaatat ggaactaacc attccaattt tcgctacgaa
ttccgactgc gagcttattg ttaaggcaat gcaaggtctc
ctaaaagatg gaaacccgat tccctcagca atcgcagcaa
actccggcat ctactaatag acgccggcca ggcttcacat
atgtattgtt ctttctaccc tccagatgag gatgaagaag
aaggtgattg tgaagaagaa gagtttgagc aaggcttcta
cgcagaaggg agcagaggcg gcagtcaagc ctcttctcgt
tcctcatcac gtagtcgcaa cagttcaaga aattcaactc
caggcagatg gtagctggca cataaaccggagctctcaat
tgcaatgaca gcttgcaaac gggtttcctc gcctccctgt
tttacaccca cagcttcaac agctctggct gccccgagcg
cttgtcttcc tgccg.

The MS2-CoV/HCV gene was inserted into a prokaryotic expression vector pET-28b to construct a pET-28b-MS2-CoV/HCV recombinant plasmid (Seq ID NO.12) as shown below sequence.

(Seq ID NO. 12)
agatctcgat cccgcgaaat taatacgact cactataggg
gaattgtgag cggataacaa ttcccctcta gaaataattt
tgtttaactt ttaagaagga gatataccat ggtggctatc
gctgtaggta gccggaattc cattcctagg aggtttgacc
tgtgcgagct tttagtaccc ttgataggga gaacgagacc
ttcgtcccct ccgttcgcgt ttacgcggac ggtgagactg
aagataactc attctcttta aaatatcgtt cgaactggac
tcccggtcgt tttaactcga ctggggccaa aacgaaacag
tggcactacc cctctccgta ttcacggggg gcgttaagtg
tcacatcgat agatcaaggt gcctacaagc gaagtgggtc
atcgtggggt cgcccgtacg aggagaaagc cggtttcggc
ttctccctcg acgcacgctc ctgctacagc ctcttccctg
taagccaaaa cttgacttac atcgaagtgc cgcagaacgt
tgcgaaccgg gcgtcgaccg aagtcctgca aaaggtcacc
cagggtaatt ttaaccttgg tgttgcttta gcagaggcca
ggtcgacagc ctcacaactc gcgacgcaaa ccattgcgct
cgtgaaggcg tacactgccg ctcgtcgcgg taattggcgc
caggcgctcc gctaccttgc cctaaacgaa gatcgaaagt
ttcgatcaaa acacgtggcc ggcaggtggt tggagttgca
gttcggttgg ttaccactaa tgagtgatat ccagggtgca
tatgagatgc ttacgaaggt tcaccttcaa gagtttcttc
ctatgagagc cgtacgtcag gtcggtacta acatcaagtt
agatggccgt ctgtcgtatc cagctgcaaa cttccagaca
acgtgcaaca tatcgcgacg tatcgtgata tggttttaca
taaacgatgc acgtttggca tggttgtcgt ctctaggtat
cttgaaccca ctaggtatag tgtgggaaaa ggtgcctttc
tcattcgttg tcgactggct cctacctgta ggtaacatgc
tcgagggcct tacggccccc gtgggatgct cctacatgtc
aggaacagtt actgacgtaa taacgggtga gtccatcata
agcgttgacg ctccctacgg gtggactgtg gagagacagg
gcactgctaa ggcccaaatc tcagccatgc atcgaggggt
acaatccgta tggccaacaa ctggcgcgta cgtaaagtct
cctttctcga tggtccatac cttagatgcg ttagcattaa
tcaggcaacg gctctctaga tagagccctc aaccggagtt
tgaagcatgg cttctaactt tactcagttc gttctcgtcg
acaatggcgg aactggcgac gtgactgtcg ccccaagcaa
cttcgctaac ggggtcgctg aatggatcag ctctaactcg
cgttcacagg cttacaaagt aacctgtagc gttcgtcaga
gctctgcgca gaatcgcaaa tacaccatca aagtcgaggt
gcctaaagtg gcaacccaga ctgttggtgg tgtagagctt
cctgtagccg catggcgttc gtacttaaat atggaactaa
ccattccaat tttcgctacg aattccgact gcgagcttat
tgttaaggca atgcaaggtc tcctaaaaga tggaaacccg
attccctcag caatcgcagc aaactccggc atctactaat
agacgccggc caggcttcac atatgtattg ttctttctac
cctccagatg aggatgaaga agaaggtgat tgtgaagaag
aagagtttga gcaaggcttc tacgcagaag ggagcagagg
cggcagtcaa gcctcttctc gttcctcatc acgtagtcgc
aacagttcaa gaaattcaac tccaggcaga tggtagctgg
cacataaacc ggaccgctct caattgcaat gacagcttgc
aaacgggttt cctcgcctcc ctgttttaca cccacagctt
caacagctct ggctgccccg agcgcttgtc ttcctgccgg
aattcggctt cacatatgta ttgttctttc taccctccag
atgaggatga agaagaaggt gattgtgaag aagaagagtt
tgagcaaggc ttctacgcag aagggagcag aggcggcagt
caagcctctt ctcgttcctc atcacgtagt cgcaacagtt
caagaaattc aactccaggc agatggtagc tggcacataa
accggaccgc tctcaattgc aatgacagct tgcaaacggg
tttcctcgcc tccctgtttt acacccacag cttcaacagc
tctggctgcc ccgagcgctt gtcttcctgc cgaagcttgc
ggccgcctcg agcaccacca ccactgagat ccggctgcta
acaaagcccg aaaggaagct gagttggctg ctgccaccgc
tgagcaataa ctagcataac cccttggggc ctctaaacgg
gtcttgaggg gttttttg

The recombinant plasmid pET-28b-MS2-CoV/HCV was transformed into E. coli competent cells, a plasmid was extracted, double enzyme digestion identification was conducted, induced culture was conducted by IPTG, then expression was identified by SDS-PAGE, and purification was conducted to obtain CoV/HCV RNA (Seq ID NO.10), i.e., a pseudotype virus standard.

The SARS-CoV-2 content of the pseudotype virus stock solution was subjected to calibrated detection by HCV.
1) The pseudotype virus stock solution was diluted E2/E3/E4/E5/E6 times with normal saline respectively to obtain different concentrations of CoV/HCV RNA.
2) The diluted CoV/HCV RNA was subjected to nucleic acid extraction, and PCR computer detection, and calibration concentrations thereof were verified with an HCV standard.

Result showing:

TABLE 8
Detection results of concentrations of
pseudotype virus CoV/HCV RNA

| | CoV | CoV | CoV | CoV | CoV |
|---|---|---|---|---|---|
| | standard | standard | standard | standard | standard |
| | stock | stock | stock | stock | stock |
| | solution | solution | solution | solution | solution |
| Name | (E2) | (E3) | (E4) | (E5) | (E6) |
| Detec- | 2.7*10⁹ | 9.45*10⁷ | 1.08*10⁶ | 9.6*10⁶ | 8.1*10⁵ |
| tion | copies/mL | copies/mL | copies/mL | copies/mL | copies/mL |
| concen- | | | | | |
| trations | | | | | |
| Dilution | 100 | 1000 | 10000 | 100000 | 1000000 |
| times | | | | | |

3) According to the obtained CoV/HCV RNA concentrations, diluting was conducted to 106 copies/mL, 105 copies/mL, 104 copies/mL, 103 copies/mL, 102 copies/mL, and 10 copies/mL.

### Embodiment 7

### Accuracy Inspection

By using the detection system of Kit 1 configured in Embodiment 1 and using the pseudotype virus CoV/HCV RNA prepared and calibrated in Embodiment 2 as a positive standard, samples of SARS-CoV-2 that have been confirmed as positive or negative samples were detected. Detection results were shown in Table 8. For samples having positive results, there were S1, S2, S4, S7, S8, S9 and S10, and copy numbers of the SARS-CoV-2 (see Table 9) were obtained according to standard curves (FIG. 1), showing that negative and undetected samples were S3, S5 and S6, and a copy number of the SARS-CoV-2 thereof was 0. The detection results were completely consistent with pre-confirmed positive or negative results, which proves that the detection accuracy of this detection system is very high.

TABLE 9
Detection of samples for SARS-CoV-2 that have been confirmed as
positive or negative samples

| Sample | SARS-CoV-2 detection results | | SARS-CoV-2 |
|---|---|---|---|
| information | ORF1ab | N | copy number |
| S1 | 21.54 | 22.01 | 9.1*10⁵ copies/mL |
| S2 | 20.14 | 20.11 | 1.3*10⁶ copies/mL |
| S3 | Undetermined | Undetermined | 0 |
| S4 | 27.174 | 28.91 | 1.37*10⁴ copies/mL |
| S5 | Undetermined | Undetermined | 0 |
| S6 | Undetermined | Undetermined | 0 |
| S7 | 28.35 | 29.07 | 1.26*10⁴ copies/mL |
| S8 | 24.53 | 24.47 | 7.24*10⁴ copies/mL |
| S9 | 29.95 | 30.06 | 1.03*10⁴ copies/mL |
| S10 | 28.82 | 29.11 | 1.19*10⁴ copies/mL |

### Embodiment 8

### Specificity Inspection

By using the detection system of Kit 1 configured in Embodiment 1 and using the pseudotype virus CoV/HCV RNA prepared and calibrated in Embodiment 2 as a positive standard, 26 kinds of viruses and bacteria were detected to synthesize plasmid samples of SARS-CoV-2 target sequences as a positive control, and a TE buffer was used as a negative control. Detection results were shown in Table 10.

TABLE 10
Detection results of 26 kinds of
viruses and bacteria

| | | ORF1ab- | SARS- | | SARS- |
|---|---|---|---|---|---|
| | | Ct | CoV-2 | N-Ct | CoV-2 |
| | Sample | value- | copy | value- | copy |
| Pathogen | information | (FAM) | number | (ROX) | number |
| Measles | National | Undeter- | 0 | Undeter- | 0 |
| virus | Institutes for | mined | | mined | |
| | Food and Drug | | | | |
| | Control | | | | |
| Mumps | National | Undeter- | 0 | Undeter- | 0 |
| virus | Institutes for | mined | | mined | |
| | Food and Drug | | | | |
| | Control | | | | |
| Rubella | National | Undeter- | 0 | Undeter- | 0 |
| virus | Institutes for | mined | | mined | |
| | Food and Drug | | | | |
| | Control | | | | |
| Pseudomonas | National | Undeter- | 0 | Undeter- | 0 |
| aeruginosa | Institutes for | mined | | mined | |
| | Food and Drug | | | | |
| | Control | | | | |
| Bordetella | Guangdong | Undeter- | 0 | Undeter- | 0 |
| pertussis | Microbial | mined | | mined | |
| | Culture | | | | |
| | Collection | | | | |
| | Center | | | | |
| Epstein- | Guangzhou | Undeter- | 0 | Undeter- | 0 |
| Barr virus | BDS Biological | mined | | mined | |
| | Technology | | | | |
| | Co., Ltd. | | | | |
| EV71 | Guangzhou | Undeter- | 0 | Undeter- | 0 |
| | BDS Biological | mined | | mined | |
| | Technology | | | | |
| | Co., Ltd. | | | | |
| CA16 | Guangzhou | Undeter- | 0 | Undeter- | 0 |
| | BDS Biological | mined | | mined | |
| | Technology | | | | |
| | Co., Ltd. | | | | |
| Mycoplasma | Guangzhou | Undeter- | 0 | Undeter- | 0 |
| pneumonia | BDS Biological | mined | | mined | |
| | Technology | | | | |
| | Co., Ltd. | | | | |
| Influenza A | National | Undeter- | 0 | Undeter- | 0 |
| (H1N1) virus | Institutes for | mined | | mined | |
| (2009) | Food and Drug | | | | |
| | Control | | | | |
| Influenza B | National | Undeter- | 0 | Undeter- | 0 |
| virus | Institutes for | mined | | mined | |
| Yamagata | Food and Drug | | | | |
| | Control | | | | |
| E. coli | Guangdong | Undeter- | 0 | Undeter- | 0 |
| | Microbial | mined | | mined | |
| | Culture | | | | |
| | Collection | | | | |
| | Center | | | | |
| Staph- | Guangdong | Undeter- | 0 | Undeter- | 0 |
| ylococcus | Microbial | mined | | mined | |
| aureus | Culture | | | | |
| | Collection | | | | |
| | Center | | | | |
| Candida | Guangdong | Undeter- | 0 | Undeter- | 0 |
| albicans | Microbial | mined | | mined | |
| | Culture | | | | |
| | Collection | | | | |
| | Center | | | | |
| Streptococcus | Guangdong | Undeter- | 0 | Undeter- | 0 |
| pneumoniae | Microbial | mined | | mined | |
| | Culture | | | | |
| | Collection | | | | |
| | Center | | | | |
| Respiratory | Shaoxing | Undeter- | 0 | Undeter- | 0 |
| syncytial | Center for | mined | | mined | |
| virus | Disease Control | | | | |
| | and Prevention | | | | |
| Influenza B | National | Undeter- | 0 | Undeter- | 0 |
| virus Victoria | Institutes for | mined | | mined | |
| | Food and Drug | | | | |
| | Control | | | | |
| Seasonal | National | Undeter- | 0 | Undeter- | 0 |
| H1N1 | Institutes for | mined | | mined | |
| | Food and Drug | | | | |
| | Control | | | | |
| Seasonal | National | Undeter- | 0 | Undeter- | 0 |
| H3N2 | Institutes for | mined | | mined | |
| | Food and Drug | | | | |
| | Control | | | | |
| Influenza A | National | Undeter- | 0 | Undeter- | 0 |
| (H7N9) virus | Institutes for | mined | | mined | |
| | Food and Drug | | | | |
| | Control | | | | |
| Aspergillus | Guangdong | Undeter- | 0 | Undeter- | 0 |
| fumigatus | Microbial | mined | | mined | |
| | Culture | | | | |
| | Collection | | | | |
| | Center | | | | |
| Klebsiella | Guangdong | Undeter- | 0 | Undeter- | 0 |
| Pneumoniae | Microbial | mined | | mined | |
| | Culture | | | | |
| | Collection | | | | |
| | Center | | | | |
| Cryptococcus | Guangdong | Undeter- | 0 | Undeter- | 0 |
| neoformans | Microbial | mined | | mined | |
| | Culture | | | | |
| | Collection | | | | |
| | Center | | | | |
| Parainfluenza | Shaoxing | Undeter- | 0 | Undeter- | 0 |
| virus | Center for | mined | | mined | |
| | Disease Control | | | | |
| | and Prevention | | | | |
| Human | Shaoxing | Undeter- | 0 | Undeter- | 0 |
| metapneumo- | Center for | mined | | mined | |
| virus | Disease Control | | | | |
| | and Prevention | | | | |
| Rhinovirus | Shaoxing | Undeter- | 0 | Undeter- | 0 |
| | Center for | mined | | mined | |
| | Disease | | | | |
| | Control and | | | | |
| | Prevention | | | | |
| Positive | / | 21.77 | 8.6* | 21.54 | 9.1* |
| control | | | 10⁵ | | 10⁵ |
| | | | copies/ | | copies/ |
| | | | mL | | mL |
| Negative | / | Undeter- | 0 | Undeter- | 0 |
| control | | mined | | mined | |

It can be seen from Table 10 that except for a positive control, results of other pathogen templates are all negative, indicating that the specificity of the detection system determined by the present invention is very good, and is 100%.

The present invention as shown and set forth in this text may be achieved in case of lacking any element and limitation disclosed herein specifically. Terms and expression methods used herein are used for description, but not for limitation. Further, it is undesired that any equivalent of the features or a portion thereof as shown or set forth herein is excluded in the use of these terms and expression methods; moreover, a person skilled in the art should realize that various modifications are feasible within the scope of the present invention. Therefore, it should be understood that the present invention is disclosed through various examples and optional features; but any amendment and variation on the concept herein can be used by a person skilled in the art. Moreover, these amendments and variations should be construed as falling within the scope of claims of the present invention.

Articles, patents, patent applications set forth or disclosed herein, as well as all other documents and contents of the electronically available information should be included herein in full text for reference to some extent, just as each individual publication is specifically and separately pointed out for reference. The Applicant reserves the right to incorporate any and all materials and information from this article, patent, patent application or other documents into the present application.

1. A kit for quantitatively detecting a copy number of Severe acute respiratory syndrome coronavirus 2 (SARS-CoV-2), wherein the kit comprises primers and probes, and the primers and the probes are respectively as follows: a first set: a sequence of a forward primer corresponding to an ORF1ab gene as shown in Seq ID NO.1 in a sequence listing; a sequence of a reverse primer corresponding to the ORF1ab gene as shown in Seq ID NO.2 in the sequence listing; and a sequence of a probe corresponding to the ORF1ab gene as shown in Seq ID NO.3 in the sequence listing.

2. The kit as claimed in claim 1, wherein the kit further comprises a second set of primers and probes, and the second set of primers and probes are as follows: a sequence of a forward primer corresponding to an N gene as shown in Seq ID NO.4 in the sequence listing; a sequence of a reverse primer corresponding to the N gene as shown in Seq ID NO.5 in the sequence listing; and a sequence of a probe corresponding to the N gene as shown in Seq ID NO.6 in the sequence listing.

3. The kit as claimed in claim 2, wherein the kit further comprises a pseudotype virus standard containing SARS-CoV-2 and HCV gene fragments.

4. The kit as claimed in claim 3, wherein a gene of the pseudotype virus standard contains a target gene CoV/HCV RNA synthesized by connecting gene sequences of ORF1ab and N conserved regions of the SARS-CoV-2 and a conserved region of HCV in series, the gene sequence of the ORF1ab conserved region being shown in Seq ID NO.7, the gene sequence of the N conserved region being shown in Seq ID NO.8, the gene sequence of the conserved region of HCV being shown in Seq ID NO.9, and a sequence of the CoV/HCV RNA being shown in Seq ID NO.10 in the sequence listing.

5. The kit as claimed in claim 4, wherein a preparation method of the pseudotype virus standard is as follows: connecting gene sequences of an MS2 phage, the ORF1ab and N conserved regions of the SARS-CoV-2 and the conserved region of HCV in series and synthesizing a target gene MS2-CoV/HCV; inserting the MS2-CoV/HCV gene into a prokaryotic expression vector pET-28b to construct a pET-28b-MS2-CoV/HCV recombinant plasmid; transforming the recombinant plasmid into E. coli competent cells, extracting a plasmid, conducting double enzyme digestion identification, conducting induced culture by IPTG, then identifying expression by SDS-PAGE, and conducting purification to obtain CoV/HCV RNA, sequences of the MS2-CoV/HCV and the pET-28b-MS2-CoV/HCV being shown in Seq ID NO.11 and Seq ID NO.12 in the sequence listing.

6. The kit as claimed in claim 2, wherein the kit further comprises a third set of internal standard primers and probes, and the third set of internal standard primers and probes are as follows: a sequence of a forward primer corresponding to a β-Globin gene as shown in Seq ID NO.13 in the sequence listing; a sequence of a reverse primer corresponding to the β-Globin gene as shown in Seq ID NO.14 in the sequence listing; a sequence of a probe corresponding to the β-Globin gene as shown in Seq ID NO.15 in the sequence listing; the three sets of probes further comprise fluorescence generating genes and fluorescence quenching genes, a fluorescence generating gene of the probe corresponding to the ORF1ab gene being FAM, and a fluorescence quenching gene thereof being BHQ1; a fluorescence generating gene of the probe corresponding to the N gene being ROX, and a fluorescence quenching gene thereof being BHQ1; and a fluorescence generating gene of the probe corresponding to the β-Globin gene being VIC, and a fluorescence quenching gene thereof being BHQ1.

7. The kit as claimed in claim 1, wherein the kit further comprises a lysis solution, a washing solution, an eluent, a proteinase K, and magnetic beads.

8. The kit as claimed in claim 1, wherein the kit further comprises a reverse transcriptase, a DNA polymerase, dNTPs, a PCR buffer, a primer, and a probe.

9. The kit as claimed in claim 8, wherein a usage amount of the reverse transcriptase is 50 to 200 U/reaction; and a usage amount of the DNA polymerase is 1 to 8 U/reaction.

10. The kit as claimed in claim 8, wherein the dNTPs are selected from a combination of dATP, dGTP, dCTP and dUTP, a combination ratio is 1:1:1:1 or 1:1:1:2, and a concentration is 10 to 25 mmol/L.

11. The kit as claimed in claim 8, wherein a concentration of magnesium ions in the PCR buffer is 50 to 100 mmol/L.

12. The kit as claimed in claim 8, wherein a concentration of the primer is 0.2 to 1 μmol/L.

13. The kit as claimed in claim 8, wherein a concentration of the probe is 0.05 to 0.1 μmol/L.

14. A kit for extraction-free nucleic acid detection of Severe acute respiratory syndrome coronavirus 2 (SARS-CoV-2), comprising an nCoV reaction solution, a PCR enhancer, an nCoV positive quality control product and an nCoV negative quality control product; and wherein the nCoV reaction solution comprises a primer probe, a DNA polymerase, a reverse transcriptase, a UNG enzyme, a nucleoside triphosphate, magnesium ions, Tris-HCl, KCl, MgCl₂ and TritonX-100; wherein the primer probe comprises primers and probes designed for conserved sequences of an ORF1ab gene and an N gene of the SARS-CoV-2, as well as primers and probes for a β-Globin gene as an internal standard.

15. (canceled)

16. (canceled)

17. The kit as claimed in claim 14, wherein sequences of ORF1ab gene-specific forward and reverse primers are respectively 5′-GGCTTCACATATGTATTGTTC-3′ and 5′-GCTCAAACTCTTCTTCTTCAC-3′; a sequence of an ORF1ab gene-specific probe is 5′-TCACCTTCTTCTTCATCCTCATCTGG-3′, and two ends of the probe are respectively labeled with a fluorescence generating group FAM and a fluorescence quenching group BHQ1.

18. The kit as claimed in claim 14, wherein sequences of N gene-specific forward (upstream) and reverse (downstream) primers are respectively 5′-AAGGCTTCTACGCAGAAG-3′ and 5′-GCTGCCTGGAGTTGAATTTC-3′; a sequence of an N gene-specific probe is 5′-AGCCTCTTCTCGTTCCTCATCAC-3′, and two ends of the probe are respectively labeled with a fluorescence generating group ROX and a fluorescence quenching group BHQ2.

19. The kit as claimed in claim 14, wherein sequences of β-Globin gene-specific forward and reverse primers are respectively 5′-CTGAGGGTTTGAAGTCCA-3′ and 5′-TCTGCCCTGACTTTTATG-3′; a sequence of a β-Globin gene-specific probe is 5′-CTCCTAAGCCAGTGCCAGAAGA-3′, and two ends of the probe are respectively labeled with a fluorescence generating group VIC and a fluorescence quenching group BHQ1.

20. The kit as claimed in claim 14, wherein an optimal concentration of the DNA polymerase in the nCoV reaction solution is 2 U/reaction, a usage amount of the reverse transcriptase is 1.5 U/reaction, and a usage amount of the UNG enzyme is 0.2 U/reaction; an optimal concentration of dNTPs is 2 mmol/L, and 5 mmol/L Tris-HCl, 10 mmol/L MgCl2 and 30 mmol/L KC1 are further comprised.

21. The kit as claimed in claim -1414, wherein the PCR enhancer comprises 1% to 6% formamide, 0.01% to 0.2% glycerol, and 1 to 5 mg/ml BSA, functioning to improve activity of the DNA polymerase and increase heat resistance of the reverse transcriptase.

22. The kit as claimed in claim 14, wherein the positive quality control product is a pUC57 vector plasmid containing an inserted SARS-CoV-2-specific conserved sequence; and the negative quality control product is a TE buffer containing a human house-keeping gene β-Globin gene sequence.


SEQUENCE LISTING (42 sequences)

SEQ ID NO: 1 - DNA, 19 bp, Artificial Sequence
  synthesized
tttccactgttttcgtgcc

SEQ ID NO: 2 - DNA, 19 bp, Artificial Sequence
  synthesized
gacccatagtagcgcagag

SEQ ID NO: 3 - DNA, 28 bp, Artificial Sequence
  synthesized
cagttcctcttcacataatcgccccgag

SEQ ID NO: 4 - DNA, 21 bp, Artificial Sequence
  synthesized
gtgctgtttcctttgccgata

SEQ ID NO: 5 - DNA, 28 bp, Artificial Sequence
  synthesized
cagttcctcttcacataatcgccccgag

SEQ ID NO: 6 - DNA, 28 bp, Artificial Sequence
  synthesized
acagtgttatttggtgcagctcgtggtt

SEQ ID NO: 7 - DNA, 80 bp, Artificial Sequence
  N conserved region of Sars-CoV-2
ggcttcacatatgtattgttctttctaccctccagatgaggatgaagaagaaggtgattgtgaagaagaagagtttgagc

SEQ ID NO: 8 - DNA, 97 bp, Artificial Sequence
  N conerved region of Sars-CoV-2
aaggcttctacgcagaagggagcagaggcggcagtcaagcctcttctcgttcctcatcacgtagtcgcaacagttcaagaaattcaactccaggcag

SEQ ID NO: 9 - DNA, 130 bp, Artificial Sequence
  conserved region of HCV
atggtagctggcacataaaccggaccgctctcaattgcaatgacagcttgcaaacgggtttcctcgcctccctgttttacacccacagcttcaacagctctggctgccccgagcgcttgtcttcctgccg

SEQ ID NO: 10 - DNA, 307 bp, Artificial Sequence
  Cov/HCV
ggcttcacatatgtattgttctttctaccctccagatgaggatgaagaagaaggtgattgtgaagaagaagagtttgagcaaggcttctacgcagaagggagcagaggcggcagtcaagcctcttctcgttcctcatcacgtagtcgcaacagttcaagaaattcaactccaggcagatggtagctggcacataaaccggaccgctctcaattgcaatgacagcttgcaaacgggtttcctcgcctccctgttttacacccacagcttcaacagctctggctgccccgagcgcttgtcttcctgccg

SEQ ID NO: 11 - DNA, 1965 bp, Artificial Sequence
  MS@-COV/HCV
tggctatcgctgtaggtagccggaattccattcctaggaggtttgacctgtgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccaaaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcatatgagatgcttacgaaggttcaccttcaagagtttcttcctatgagagccgtacgtcaggtcggtactaacatcaagttagatggccgtctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccaggcttcacatatgtattgttctttctaccctccagatgaggatgaagaagaaggtgattgtgaagaagaagagtttgagcaaggcttctacgcagaagggagcagaggcggcagtcaagcctcttctcgttcctcatcacgtagtcgcaacagttcaagaaattcaactccaggcagatggtagctggcacataaaccggagctctcaattgcaatgacagcttgcaaacgggtttcctcgcctccctgttttacacccacagcttcaacagctctggctgccccgagcgcttgtcttcctgccg

SEQ ID NO: 12 - DNA, 2538 bp, Artificial Sequence
  PET-28b-MS2-COV/HCV
agatctcgatcccgcgaaattaatacgactcactataggggaattgtgagcggataacaattcccctctagaaataattttgtttaacttttaagaaggagatataccatggtggctatcgctgtaggtagccggaattccattcctaggaggtttgacctgtgcgagcttttagtacccttgatagggagaacgagaccttcgtcccctccgttcgcgtttacgcggacggtgagactgaagataactcattctctttaaaatatcgttcgaactggactcccggtcgttttaactcgactggggccaaaacgaaacagtggcactacccctctccgtattcacggggggcgttaagtgtcacatcgatagatcaaggtgcctacaagcgaagtgggtcatcgtggggtcgcccgtacgaggagaaagccggtttcggcttctccctcgacgcacgctcctgctacagcctcttccctgtaagccaaaacttgacttacatcgaagtgccgcagaacgttgcgaaccgggcgtcgaccgaagtcctgcaaaaggtcacccagggtaattttaaccttggtgttgctttagcagaggccaggtcgacagcctcacaactcgcgacgcaaaccattgcgctcgtgaaggcgtacactgccgctcgtcgcggtaattggcgccaggcgctccgctaccttgccctaaacgaagatcgaaagtttcgatcaaaacacgtggccggcaggtggttggagttgcagttcggttggttaccactaatgagtgatatccagggtgcatatgagatgcttacgaaggttcaccttcaagagtttcttcctatgagagccgtacgtcaggtcggtactaacatcaagttagatggccgtctgtcgtatccagctgcaaacttccagacaacgtgcaacatatcgcgacgtatcgtgatatggttttacataaacgatgcacgtttggcatggttgtcgtctctaggtatcttgaacccactaggtatagtgtgggaaaaggtgcctttctcattcgttgtcgactggctcctacctgtaggtaacatgctcgagggccttacggcccccgtgggatgctcctacatgtcaggaacagttactgacgtaataacgggtgagtccatcataagcgttgacgctccctacgggtggactgtggagagacagggcactgctaaggcccaaatctcagccatgcatcgaggggtacaatccgtatggccaacaactggcgcgtacgtaaagtctcctttctcgatggtccataccttagatgcgttagcattaatcaggcaacggctctctagatagagccctcaaccggagtttgaagcatggcttctaactttactcagttcgttctcgtcgacaatggcggaactggcgacgtgactgtcgccccaagcaacttcgctaacggggtcgctgaatggatcagctctaactcgcgttcacaggcttacaaagtaacctgtagcgttcgtcagagctctgcgcagaatcgcaaatacaccatcaaagtcgaggtgcctaaagtggcaacccagactgttggtggtgtagagcttcctgtagccgcatggcgttcgtacttaaatatggaactaaccattccaattttcgctacgaattccgactgcgagcttattgttaaggcaatgcaaggtctcctaaaagatggaaacccgattccctcagcaatcgcagcaaactccggcatctactaatagacgccggccaggcttcacatatgtattgttctttctaccctccagatgaggatgaagaagaaggtgattgtgaagaagaagagtttgagcaaggcttctacgcagaagggagcagaggcggcagtcaagcctcttctcgttcctcatcacgtagtcgcaacagttcaagaaattcaactccaggcagatggtagctggcacataaaccggaccgctctcaattgcaatgacagcttgcaaacgggtttcctcgcctccctgttttacacccacagcttcaacagctctggctgccccgagcgcttgtcttcctgccggaattcggcttcacatatgtattgttctttctaccctccagatgaggatgaagaagaaggtgattgtgaagaagaagagtttgagcaaggcttctacgcagaagggagcagaggcggcagtcaagcctcttctcgttcctcatcacgtagtcgcaacagttcaagaaattcaactccaggcagatggtagctggcacataaaccggaccgctctcaattgcaatgacagcttgcaaacgggtttcctcgcctccctgttttacacccacagcttcaacagctctggctgccccgagcgcttgtcttcctgccgaagcttgcggccgcctcgagcaccaccaccactgagatccggctgctaacaaagcccgaaaggaagctgagttggctgctgccaccgctgagcaataactagcataaccccttggggcctctaaacgggtcttgaggggttttttg

SEQ ID NO: 13 - DNA, 27 bp, Artificial Sequence
  synthesized
ttgcgtgattatttagctgactatgac

SEQ ID NO: 14 - DNA, 25 bp, Artificial Sequence
  synthesized
tcctgtaccaccaacattaatagca

SEQ ID NO: 15 - DNA, 21 bp, Artificial Sequence
  synthesized
tcactgccggcccattcatgg

SEQ ID NO: 16 - DNA, 20 bp, Artificial Sequence
  synthesized
actggactttattgacacta

SEQ ID NO: 17 - DNA, 18 bp, Artificial Sequence
  synthesized
cgtgtaccaagcaatttc

SEQ ID NO: 18 - DNA, 16 bp, Artificial Sequence
  synthesized
atactgctgccgtgaa

SEQ ID NO: 19 - DNA, 18 bp, Artificial Sequence
  synthesized
ggatcaagaatcctttgg

SEQ ID NO: 20 - DNA, 17 bp, Artificial Sequence
  synthesized
agcacaagttgtaggta

SEQ ID NO: 21 - DNA, 20 bp, Artificial Sequence
  synthesized
actgccgttgccacatagat

SEQ ID NO: 22 - DNA, 16 bp, Artificial Sequence
  synthesized
cgtctacaagctggta

SEQ ID NO: 23 - DNA, 17 bp, Artificial Sequence
  synthesized
ttccggtgtaactgtta

SEQ ID NO: 24 - DNA, 25 bp, Artificial Sequence
  synthesized
tgcctgccaattcaactgtattatc

SEQ ID NO: 25 - DNA, 18 bp, Artificial Sequence
  synthesized
actccaggcagcagtagg

SEQ ID NO: 26 - DNA, 18 bp, Artificial Sequence
  synthesized
agcagcagcaaagcaaga

SEQ ID NO: 27 - DNA, 24 bp, Artificial Sequence
  synthesized
catcaccgccattgccagccattc

SEQ ID NO: 28 - DNA, 18 bp, Artificial Sequence
  synthesized
aggcagcagtaggggaac

SEQ ID NO: 29 - DNA, 15 bp, Artificial Sequence
  synthesized
caagcagcaaagcaa

SEQ ID NO: 30 - DNA, 24 bp, Artificial Sequence
  synthesized
catcaccgccattgccagccattc

SEQ ID NO: 31 - DNA, 19 bp, Artificial Sequence
  synthesized
gctgtttcctttgccgata

SEQ ID NO: 32 - DNA, 16 bp, Artificial Sequence
  synthesized
agcagcagcaaagcaa

SEQ ID NO: 33 - DNA, 24 bp, Artificial Sequence
  synthesized
agtgttatttggtgcagctcgtgg

SEQ ID NO: 34 - DNA, 21 bp, Artificial Sequence
  synthesized
ggcttcacatatgtattgttc

SEQ ID NO: 35 - DNA, 21 bp, Artificial Sequence
  synthesized
gctcaaactcttcttcttcac

SEQ ID NO: 36 - DNA, 26 bp, Artificial Sequence
  synthesized
tcaccttcttcttcatcctcatctgg

SEQ ID NO: 37 - DNA, 18 bp, Artificial Sequence
  synthesized
aaggcttctacgcagaag

SEQ ID NO: 38 - DNA, 20 bp, Artificial Sequence
  synthesized
gctgcctggagttgaatttc

SEQ ID NO: 39 - DNA, 23 bp, Artificial Sequence
  synthesized
agcctcttctcgttcctcatcac

SEQ ID NO: 40 - DNA, 18 bp, Artificial Sequence
  synthesized
ctgagggtttgaagtcca

SEQ ID NO: 41 - DNA, 18 bp, Artificial Sequence
  synthesized
tctgccctgacttttatg

SEQ ID NO: 42 - DNA, 22 bp, Artificial Sequence
  synthesized
ctcctaagccagtgccagaaga